(19) United States
(12) Patent Application Publication
(10) Pub. No.: US 2024/0277716 A1
(43) Pub. Date: Aug. 22, 2024

(54) Suppressive T Cell Populations and Methods of Cancer Immunotherapy

(71) Applicant: La Jolla Institute for Immunology, La Jolla, CA (US)

(72) Inventors: Pandurangan Vijayanand, La Jolla, CA (US); Christian Ottensmeier, La Jolla, CA (US); Simon Eschweiler, La Jolla, CA (US); Mitchell Kronenberg, La Jolla, CA (US)

(21) Appl. No.: 18/564,913

(22) PCT Filed: Jun. 3, 2022
(86) PCT No.: PCT/US2022/032124

§ 371 Date: Nov. 28, 2023

Related U.S. Application Data
(60) Provisional application No. 63/197,060, filed on Jun. 4, 2021.
(60) Provisional application No. 63/235,496, filed on Aug. 20, 2021.

(51) Int. Cl. A61K 31/52; A61K 31/519; A61K 39/00; A61K 39/395; A61P 35/00; A61P 37/06; C07K 16/28; G01N 33/569

(52) U.S. Cl. CPC A61K 31/52; A61K 31/519; A61K 39/39558; A61P 35/00; A61P 37/06; C07K 16/2818; G01N 33/56972; A61K 2039/505; G01N 2800/52

(57) ABSTRACT

The present invention includes methods of treating cancer in a patient, the method comprising the steps of: (a) providing or obtaining a sample from a patient; (b) determining at least one of a level or activity of at least one of: T follicular regulatory (T_{FR}), ST2 Treg, highly suppressive Treg, activated Treg, or effector Treg cells in the sample; (c) comparing the at least one level or activity of the T cells in the sample to a level or activity of the T cells in a reference sample, respectively, for a specific tumor type or a healthy subject; and (d) if the patient has the at least one of the high level or activity of the T cells systemically as compared to the reference sample, then administering a cancer therapy to the patient that comprises a modified dosage or administration of a Phosphoinositide 3-kinase (PI3K) inhibitor.

### CROSS-REFERENCE TO RELATED APPLICATIONS

This application claims priority to U.S. Provisional Application Ser. No. 63/197,060, filed Jun. 4, 2021, and U.S. Provisional Application Ser. No. 63/235,496, filed Aug. 20, 2021, the entire contents of each of which are incorporated herein by reference.

### TECHNICAL FIELD OF THE INVENTION

The present invention relates in general to the field of T follicular Regulatory (T_{FR}) cells, and more particularly, to novel methods for treating cancer and preventing Immune Related Adverse Effects (irAEs) through the modified administration of Phosphoinositide 3-kinases (PI3K) Inhibitors. The present invention also relates in general to the field of T follicular Regulatory (T_{FR}) cells, and more particularly, to novel methods for improving the efficacy of cancer vaccines by manipulating T_{FR} cells.

### STATEMENT OF FEDERALLY-FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with cancer and immune checkpoint therapies.

An increased density of T regulatory cells (T_{REG}) in tumors has been linked to poor survival outcomes. In non-cancer settings, T_{REG} cells have been shown to differentiate into PD-1 expressing follicular regulatory T cells (T_{FR}) that restrain germinal center responses. It was not previously known whether such differentiation also occurs in the tumor microenvironment, and if so, whether such tumor-infiltrating T_{FR} cells are molecularly distinct from T_{REG} cells or are activated by anti-PD1 therapy.

What is needed is to prevent immunosuppression caused by anti-PD-1 therapy and/or reduction of irAE frequency or severity caused by immunotherapy. Also needed is an increase to the efficacy of cancer vaccines.

### SUMMARY OF THE INVENTION

As embodied and broadly described herein, an aspect of the present disclosure relates to a method of treating cancer in a patient, the method comprising the steps of: (a) providing or obtaining a sample from the patient; (b) determining at least one of a level or activity of at least one of: T follicular regulatory (T_{FR}), ST2 Treg, highly suppressive Treg, activated Treg, or effector Treg cells in the sample; (c) comparing the at least one of level or activity of the at least one of T_{FR} cells, ST2 Treg, highly suppressive Treg, activated Treg, or effector Treg in the sample to a level or activity of T_{FR}, ST2 Treg, highly suppressive Treg, activated Treg, or effector Treg cells in a reference sample, respectively, for a specific tumor type or a healthy subject; and (d) if the patient has a high level or activity of T_{FR}, ST2 Treg, highly suppressive Treg, activated Treg, or effector Treg cells systemically as compared to the reference sample, then administering a cancer therapy to the patient that comprises a modified dosage or administration of a Phosphoinositide 3-kinase (PI3K) inhibitor. In one aspect, the modified dosage or administration of the PI3K inhibitor selectively depletes T_{FR}, ST2 Treg, highly suppressive Treg, activated Treg, or effector Treg cells preferentially in a tumor microenvironment, in tumor-draining lymph nodes, or both. In another aspect, modified dosage or administration of the PI3K inhibitor transiently depletes T_{FR}, ST2 Treg, highly suppressive Treg, activated Treg, or effector Treg cells systemically. In another aspect, the modified dosage or administration of the PI3K inhibitor preferentially depletes T_{FR}, ST2 Treg, highly suppressive Treg, activated Treg, or effector Treg cells in a tumor microenvironment. In another aspect, the PI3K inhibitor is provided in conjunction with, or followed by, an additional cancer therapy. In another aspect, the additional cancer therapy is a checkpoint inhibitor or other immunotherapy. In another aspect, the immune checkpoint inhibitor or immunotherapy comprises an antibody directed against a cellular protein selected from at least one of: PD-1, PD-L1, CTLA-4, TIM-3, BTLA, LAG-3, or TIGIT. In another aspect, the PI3K inhibitor is a selective Phosphoinositide 3-kinase δ inhibitor. In another aspect, the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114. In another aspect, the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺GITR⁺ T cells, or any combination thereof. In another aspect, the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1. In another aspect, the method further comprises isolating the T_{FR} cells from the sample prior to determining the level or activity of T_{FR} cells in the sample. In another aspect, the cancer is selected from a colorectal, a melanoma, a lung, a liver, a head and neck, and a breast cancer. In another aspect, the sample is a tumor biopsy. In another aspect, the step of determining the level or activity of T_{FR}, ST2 Treg, highly suppressive Treg, activated Treg, or effector Treg cells in the sample is performed by measuring mRNA, protein, or both.

As embodied and broadly described herein, an aspect of the present disclosure relates to a method of stratifying cancer patients to select an effective cancer treatment for administration, comprising: (a) providing or obtaining a sample from a patient; (b) determining at least one of a level or activity of T-follicular regulatory (T_{FR}), ST2 Treg, highly suppressive Treg, activated Treg, or effector Treg cells in the sample; (c) comparing the at least one of a level or activity of T_{FR}, ST2 Treg, highly suppressive Treg, activated Treg, or effector Treg cells in the sample to a level or activity of T_{FR}, ST2 Treg, highly suppressive Treg, activated Treg, or effector Treg cells in a reference sample, respectively, for a specific tumor type or a healthy subject; and (d) stratifying the patient into at least one of three groups selected from: (1) a high level or increase in the level or activity of T_{FR}, ST2 Treg, highly suppressive Treg, activated Treg, or effector Treg cells in a tumor microenvironment or tumor-draining lymph nodes, (2) a high level or activity of T_{FR}, ST2 Treg, highly suppressive Treg, activated Treg, or effector Treg cells systemically, or (3) a low level or activity of T_{FR}, ST2 Treg, highly suppressive Treg, activated Treg, or effector Treg cells in a tumor microenvironment or tumor-draining lymph node, or (4) a low level or activity of T_{FR}, ST2 Treg, highly suppressive Treg, activated Treg, or effector Treg cells systemically, when compared to the reference for the specific tumor type or the healthy subject. In one aspect, if the patient has a high level or activity of T_{FR}, ST2 Treg, highly suppressive Treg, activated Treg, or effector Treg cells systemically, then selecting a cancer treatment that does not consist of administration of a Phosphoinositide 3-kinase (PI3K) inhibitor. In another aspect, if the patient has an increase in the level or activity of T_{FR}, ST2 Treg, highly suppressive Treg, activated Treg, or effector Treg cells in the tumor microenvironment or tumor-draining lymph nodes, further comprising administering a T_{FR} cell depleting therapy capable of selectively depleting T_{FR}, ST2 Treg, highly suppressive Treg, activated Treg, or effector Treg cells preferentially in the tumor microenvironment or tumor-draining lymph nodes, or both, prior to or concurrent with administration of a cancer treatment. In another aspect, if the patient has a high level or an increase in the level or activity of T_{FR}, ST2 Treg, highly suppressive Treg, activated Treg, or effector Treg cells in the tumor microenvironment or tumor-draining lymph nodes or a high level or activity of T_{FR}, ST2 Treg, highly suppressive Treg, activated Treg, or effector Treg cells systemically, further comprising administering a T_{FR} cell depleting therapy capable of transiently depleting T_{FR}, ST2 Treg, highly suppressive Treg, activated Treg, or effector Treg cells systemically prior to or concurrent with administration of a cancer treatment. In another aspect, if the patient has a high level or an increase in the level or activity of T_{FR}, ST2 Treg, highly suppressive Treg, activated Treg, or effector Treg cells in the tumor microenvironment or tumor-draining lymph nodes, further comprising administering a T_{FR}, ST2 Treg, highly suppressive Treg, activated Treg, or effector Treg cell depleting therapy capable of preferentially depleting T_{FR}, ST2 Treg, highly suppressive Treg, activated Treg, or effector Treg cells in the tumor microenvironment or tumor-draining lymph nodes prior to or concurrent with administration of a cancer treatment. In another aspect, if the patient has a low level or decrease or low activity of T_{FR}, ST2 Treg, highly suppressive Treg, activated Treg, or effector Treg cells in the tumor microenvironment or tumor-draining lymph node, further comprising administering an immunotherapy agent or antibody. In another aspect, if the patient has a low level or decrease or low activity of T_{FR}, ST2 Treg, highly suppressive Treg, activated Treg, or effector Treg cells systemically, further comprising administering an immunotherapy agent or antibody. In another aspect, the T_{FR}, ST2 Treg, highly suppressive Treg, activated Treg, or effector Treg cell depleting therapy is provided in conjunction with, or followed by, the cancer treatment. In another aspect, the cancer treatment is a checkpoint inhibitor or other immunotherapy. In another aspect, the checkpoint inhibitor or immunotherapy comprises an antibody directed against a cellular protein selected from at least one of: PD-1, PD-L1, CTLA-4, TIM-3, BTLA, LAG-3, or TIGIT. In another aspect, the T_{FR} cell depleting therapy comprises administering one or more of the following: a modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on T_{FR} cells when compared to T_{REG} cells and other T cell populations, wherein the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off. In another aspect, the T_{FR} cell depleting therapy does not substantially reduce or eliminate T_{REGS}. In another aspect, the PI3K inhibitor is a selective Phosphoinositide 3-kinase δ inhibitor. In another aspect, the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114. In another aspect, T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺ GITR⁺ T cells, or any combination thereof. In another aspect, the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1. In another aspect, the cancer is selected from a colorectal, a melanoma, a lung, a liver, a head and neck, and a breast cancer. In another aspect, the patient sample is a tumor biopsy. In another aspect, the step of determining the level or activity of T follicular regulatory (T_{FR}), ST2 Treg, highly suppressive Treg, activated Treg, or effector Treg cells in the sample is performed by measuring mRNA, protein, or both.

As embodied and broadly described herein, an aspect of the present disclosure relates to a method of treating cancer, comprising: (a) providing or obtaining a sample from a patient; (b) determining at least one of a level or activity of at least one of: T-follicular regulatory (T_{FR}), ST2 Treg, highly suppressive Treg, activated Treg, or effector Treg cells in the sample; (c) comparing the least one of level or activity of T_{FR} cells in the sample to a level or activity of T_{FR} cells in a reference sample, respectively, for a specific tumor type or a healthy subject; (d) stratifying the patient into at least one of three groups selected from: (1) an increase in the level or activity of T_{FR}, ST2 Treg, highly suppressive Treg, activated Treg, or effector Treg cells in a tumor microenvironment or tumor-draining lymph nodes, (2) a high level or activity of T_{FR}, ST2 Treg, highly suppressive Treg, activated Treg, or effector Treg cells systemically, or (3) a low level or activity of T_{FR}, ST2 Treg, highly suppressive Treg, activated Treg, or effector Treg cells in a tumor microenvironment or tumor-draining lymph node, or (4) a low level or activity of T_{FR}, ST2 Treg, highly suppressive Treg, activated Treg, or effector Treg cells systemically, when compared to the reference for the specific tumor type or the healthy subject; and (e) administering an appropriate cancer treatment based on the stratification step of (d). In one aspect, if the patient has a high level or activity of T_{FR}, ST2 Treg, highly suppressive Treg, activated Treg, or effector Treg cells systemically, then selecting a cancer treatment that does not consist of administration of a Phosphoinositide 3-kinase (PI3K) inhibitor. In another aspect, if the patient has an increase in the level or activity of T_{FR} cells in a tumor microenvironment or tumor-draining lymph nodes, further comprising administering a T_{FR}, ST2 Treg, highly suppressive Treg, activated Treg, or effector Treg cell depleting therapy capable of selectively depleting T_{FR}, ST2 Treg, highly suppressive Treg, activated Treg, or effector Treg cells preferentially in the tumor microenvironment or tumor-draining lymph nodes, or both, prior to or concurrent with the administration of the cancer treatment. In another aspect, if the patient has an increase in the level or activity of T_{FR}, ST2 Treg, highly suppressive Treg, activated Treg, or effector Treg cells in a tumor microenvironment or tumor-draining lymph nodes or a high level or activity of T_{FR}, ST2 Treg, highly suppressive Treg, activated Treg, or effector Treg cells systemically, further comprising administering a T_{FR}, ST2 Treg, highly suppressive Treg, activated Treg, or effector Treg cell depleting therapy capable of transiently depleting T_{FR} cells systemically prior to or concurrent with the administration of the cancer treatment. In another aspect, if the patient has an increase in the level or activity of T_{FR}, ST2 Treg, highly suppressive Treg, activated Treg, or effector Treg cells in a tumor microenvironment or tumor-draining lymph nodes, further comprising administering a T_{FR}, ST2 Treg, highly suppressive Treg, activated Treg, or effector Treg cell depleting therapy capable of preferentially depleting T_{FR}, ST2 Treg, highly suppressive Treg, activated Treg, or effector Treg cells in the tumor microenvironment or tumor-draining lymph nodes prior to or concurrent with the administration of the cancer treatment. In another aspect, if the patient has a low level or decrease or low activity of T_{FR}, ST2 Treg, highly suppressive Treg, activated Treg, or effector Treg cells in a tumor microenvironment or tumor-draining lymph node, further comprising administering an immunotherapy agent or antibody. In another aspect, if the patient has a low level or decrease or low activity of T_{FR}, ST2 Treg, highly suppressive Treg, activated Treg, or effector Treg cells systemically, further comprising administering an immunotherapy agent or antibody. In another aspect, the T_{FR}, ST2 Treg, highly suppressive Treg, activated Treg, or effector Treg cell depleting therapy is provided in conjunction with, or followed by, the cancer treatment. In another aspect, the cancer treatment is a checkpoint inhibitor or other immunotherapy agent or antibody. In another aspect, the checkpoint inhibitor or immunotherapy agent comprises an antibody directed against a cellular protein selected from at least one of: PD-1, PD-L1, CTLA-4, TIM-3, BTLA, LAG-3, or TIGIT. In another aspect, the immunotherapy agent comprises an antibody directed against a cellular protein selected from at least one of: PD-1, PD-L1, TIM-3, BTLA, LAG-3, or TIGIT. In another aspect, the T_{FR} cell depleting therapy comprises administering one or more of the following: a modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on T_{FR} cells when compared to T_{REG} cells and other T cell populations, wherein the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off. In another aspect, the T_{FR} cell depleting therapy does not substantially reduce or eliminate T_{REGS}. In another aspect, the PI3K inhibitor is a selective Phosphoinositide 3-kinase δ inhibitor. In another aspect, the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114. In another aspect, the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺GITR⁺ T cells, or any combination thereof. In another aspect, the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1. In another aspect, the cancer is selected from a colorectal, a melanoma, a lung, a liver, a head and neck, and a breast cancer. In another aspect, the patient sample is a tumor biopsy. In another aspect, the step of determining the level or activity of T follicular regulatory (T_{FR}) cells in the sample is performed by measuring mRNA, protein, or both.

As embodied and broadly described herein, an aspect of the present disclosure relates to a method of treating a patient with a cancer vaccine, the method comprising the steps of: (a) providing or obtaining a sample from the patient; (b) determining at least one of a level or activity of at least one of: T follicular regulatory (T_{FR}), ST2 Treg, highly suppressive Treg, activated Treg, or effector Treg cells in the sample; (c) comparing the at least one of level or activity of T_{FR}, ST2 Treg, highly suppressive Treg, activated Treg, or effector Treg cells in the sample to a level or activity of T_{FR} cells in a reference sample, respectively, for a specific tumor type or a healthy subject; and (d) if the patient has a high level or activity of T_{FR}, ST2 Treg, highly suppressive Treg, activated Treg, or effector Treg cells systemically as compared to the reference sample, then administering a modified dosage or administration of an agent that reduces the level or activity of T_{FR}, ST2 Treg, highly suppressive Treg, activated Treg, or effector Treg cells systemically prior to, or concurrent with, administration of a cancer vaccine to the patient. In one aspect, the cancer vaccine is a tumor cell vaccine, an antigen vaccine, a dendritic cell vaccine, a DNA vaccine, an mRNA vaccine or a vector-based vaccine. In another aspect, the cancer vaccine is directed to a cancer antigen selected from at least one of: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, NY-ESO-1, LAGE-1, SSX-1, SSX-2 (HOM-MEL-40), SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RARalpha fusion protein, PTPRK, K-ras, N-ras, Triosephosphate isomerase, GnTV, Herv-K-mel, Lage-1, NA-88, /Lage-2, SP17, and TRP2-Int2, (MART-I), gp100, TRP-1, TRP-2, MAGE-1, MAGE-3, p15(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, CDK4, Mum-1, p16, TAGE, PSMA, PSCA, CT7, telomerase, 43-9F, 5T4, 791Tgp72, alpha-fetoprotein, 13HCG, BCA225, BTAA, CA 125, CA 15-3, CA 195, CA 242, CA-50, CAM43, CD68\KP1, CO-029, FGF-5, G250, Ga733, HTgp-175, M344, MA-50, MG7-Ag, MOV18, NB\170K, NY-CO-1, RCAS1, SDCCAG16, TA-90, TAAL6, TAG72, TLP, TPS; or immunogenic fragments that comprise an epitope of any of the foregoing antigens. In another aspect, the agent comprises a modified dosage or administration of a PI3K inhibitor that selectively or transiently depletes T_{FR}, ST2 Treg, highly suppressive Treg, activated Treg, or effector Treg cells preferentially in a tumor microenvironment, in tumor-draining lymph nodes, or systemically. In another aspect, the PI3K inhibitor is provided in conjunction with, or followed by, an additional cancer therapy. In another aspect, the additional cancer therapy is a checkpoint inhibitor or other immunotherapy. In another aspect, the immune checkpoint inhibitor or immunotherapy comprises an antibody directed against a cellular protein selected from at least one of: PD-1, PD-L1, CTLA-4, TIM-3, BTLA, LAG-3, or TIGIT. In another aspect, the T_{FR} cell depleting therapy comprises administering one or more of the following a modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on T_{FR} cells when compared to T_{REG} cells and other T cell populations, wherein the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off. In another aspect, the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114. In another aspect, the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺ GITR⁺ T cells, or any combination thereof. In another aspect, the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1. In another aspect, the method further comprises isolating the T_{FR}, ST2 Treg, highly suppressive Treg, activated Treg, or effector Treg cells from the sample prior to determining the level or activity of T_{FR} cells in the sample. In another aspect, the cancer is selected from a colorectal, a melanoma, a lung, a liver, a head and neck, and a breast cancer. In another aspect, the sample is a tumor biopsy. In another aspect, the step of determining the level or activity of T_{FR}, ST2 Treg, highly suppressive Treg, activated Treg, or effector Treg cells in the sample is performed by measuring mRNA, protein, or both.

As embodied and broadly described herein, an aspect of the present disclosure relates to a method of stratifying cancer patients for administration of a cancer vaccine, comprising: (a) providing or obtaining a sample from a patient; (b) determining at least one of a level or activity of T-follicular regulatory (T_{FR}), ST2 Treg, highly suppressive Treg, activated Treg, or effector Treg cells in the sample; (c) comparing the at least one of a level or activity of T_{FR}, ST2 Treg, highly suppressive Treg, activated Treg, or effector Treg cells in the sample to a level or activity of T_{FR} cells in a reference sample, respectively, for a specific tumor type or a healthy subject; and (d) stratifying the patient into at least one of three groups selected from: (1) a high level or increase in the level or activity of T_{FR}, ST2 Treg, highly suppressive Treg, activated Treg, or effector Treg cells in a tumor microenvironment or tumor-draining lymph nodes, (2) a high level or activity of T_{FR}, ST2 Treg, highly suppressive Treg, activated Treg, or effector Treg cells systemically, or (3) a low level or activity of T_{FR}, ST2 Treg, highly suppressive Treg, activated Treg, or effector Treg cells in a tumor microenvironment or tumor-draining lymph node, or (4) a low level or activity of T_{FR}, ST2 Treg, highly suppressive Treg, activated Treg, or effector Treg cells systemically, when compared to the reference for the specific tumor type or the healthy subject, wherein the cancer vaccine is administered to the patient. In one aspect, the cancer vaccine is a tumor cell vaccine, an antigen vaccine, a dendritic cell vaccine, a DNA vaccine, an mRNA vaccine or a vector-based vaccine. In another aspect, if the patient is in groups (1) or (2), the patient is also provided with a T_{FR} cell depleting therapy. In another aspect, the cancer vaccine is directed to a cancer antigen selected from at least one of: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, NY-ESO-1, LAGE-1, SSX-1, SSX-2 (HOM-MEL-40), SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RARalpha fusion protein, PTPRK, K-ras, N-ras, Triosephosphate isomerase, GnTV, Herv-K-mel, Lage-1, NA-88, /Lage-2, SP17, and TRP2-Int2, (MART-I), gp100, TRP-1, TRP-2, MAGE-1, MAGE-3, pi5(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, pi80erbB-3, c-met, nm-23H1, PSA, TAG-72-4, CA 19-9, CA 72-4, CAM 17.1, NuMa, K-ras, CDK4, Mum-1, p16, TAGE, PSMA, PSCA, CT7, telomerase, 43-9F, 5T4, 791Tgp72, alpha-fetoprotein, 13HCG, BCA225, BTAA, CA 125, CA 15-3, CA 195, CA 242, CA-50, CAM43, CD68\KP1, CO-029, FGF-5, G250, Ga733, HTgp-175, M344, MA-50, MG7-Ag, MOV18, NB\170K, NY-CO-1, RCAS1, SDCCAG16, TA-90, TAAL6, TAG72, TLP, TPS; or immunogenic fragments that comprise an epitope of any of the foregoing antigens. In another aspect, if the patient has an increase in the level or activity of T_{FR}, ST2 Treg, highly suppressive Treg, activated Treg, or effector Treg cells in the tumor microenvironment or tumor-draining lymph nodes, further comprising administering a T_{FR}, ST2 Treg, highly suppressive Treg, activated Treg, or effector Treg cell depleting therapy capable of selectively or transiently depleting T_{FR}, ST2 Treg, highly suppressive Treg, activated Treg, or effector Treg cells preferentially in the tumor microenvironment or In another aspect, if the patient has a high level or an increase in the level or activity of T_{FR}, ST2 Treg, highly suppressive Treg, activated Treg, or effector Treg cells in the tumor microenvironment or tumor-draining lymph nodes, further comprising administering a T_{FR}, ST2 Treg, highly suppressive Treg, activated Treg, or effector Treg cell depleting therapy capable of preferentially depleting T_{FR} cells in the tumor microenvironment or tumor-draining lymph nodes prior to or concurrent with administration of a cancer vaccine. In another aspect, if the patient has a low level or decrease or low activity of T_{FR}, ST2 Treg, highly suppressive Treg, activated Treg, or effector Treg cells in the tumor microenvironment or tumor-draining lymph node, further comprising administering an immunotherapy agent or antibody. In another aspect, if the patient has a low level or decrease or low activity of T_{FR} cells systemically, further comprising administering an immunotherapy agent or antibody. In another aspect, the T_{FR} cell depleting therapy is provided in conjunction with, or followed by, a cancer treatment. In another aspect, the cancer treatment is a checkpoint inhibitor or other immunotherapy. In another aspect, the checkpoint inhibitor or immunotherapy comprises an antibody directed against a cellular protein selected from at least one of: PD-1, PD-L1, CTLA-4, TIM-3, BTLA, LAG-3, or TIGIT. In another aspect, the T_{FR} cell depleting therapy comprises administering one or more of the following: a modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on T_{FR} cells when compared to T_{REG} cells and other T cell populations, wherein the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off. In another aspect, the T_{FR}, ST2 Treg, highly suppressive Treg, activated Treg, or effector Treg cell depleting therapy does not substantially reduce or eliminate T_{REGS}. In another aspect, the PI3K inhibitor is a selective Phosphoinositide 3-kinase δ inhibitor. In another aspect, the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114. In another aspect, the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺ GITR⁺ T cells, or any combination thereof. In another aspect, the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1. In another aspect, the cancer is selected from a colorectal, a melanoma, a lung, a liver, a head and neck, and a breast cancer. In another aspect, the patient sample is a tumor biopsy. In another aspect, the step of determining the level or activity of T follicular regulatory (T_{FR}) cells in the sample is performed by measuring mRNA, protein, or both.

As embodied and broadly described herein, an aspect of the present disclosure relates to a method of treating cancer with a cancer vaccine, comprising: (a) providing or obtaining a sample from a patient; (b) determining at least one of a level or activity of at least one of: T-follicular regulatory (T_{FR}), ST2 Treg, highly suppressive Treg, activated Treg, or effector Treg cells in the sample; (c) comparing the least one of level or activity of T_{FR}, ST2 Treg, highly suppressive Treg, activated Treg, or effector Treg cells in the sample to a level or activity of T_{FR} cells in a reference sample, respectively, for a specific tumor type or a healthy subject; (d) stratifying the patient into at least one of three groups selected from: (1) an increase in the level or activity of T_{FR}, ST2 Treg, highly suppressive Treg, activated Treg, or effector Treg cells in a tumor microenvironment or tumor-draining lymph nodes, (2) a high level or activity of T_{FR}, ST2 Treg, highly suppressive Treg, activated Treg, or effector Treg cells systemically, or (3) a low level or activity of T_{FR}, ST2 Treg, highly suppressive Treg, activated Treg, or effector Treg cells in a tumor microenvironment or tumor-draining lymph node, or (4) a low level or activity of T_{FR}, ST2 Treg, highly suppressive Treg, activated Treg, or effector Treg cells systemically, when compared to the reference for the specific tumor type or the healthy subject; and (e) administering the cancer vaccine based on the stratification step of (d). In one aspect, if the patient has a high level or activity of T_{FR}, ST2 Treg, highly suppressive Treg, activated Treg, or effector Treg cells systemically, then selecting a cancer vaccine that does not require treatment with a Phosphoinositide 3-kinase (PI3K) inhibitor. In another aspect, if the patient has an increase in the level or activity of T_{FR}, ST2 Treg, highly suppressive Treg, activated Treg, or effector Treg cells in a tumor microenvironment or tumor-draining lymph nodes, further comprising administering a T_{FR}, ST2 Treg, highly suppressive Treg, activated Treg, or effector Treg cell depleting therapy capable of selectively or transiently depleting T_{FR}, ST2 Treg, highly suppressive Treg, activated Treg, or effector Treg cells preferentially in the tumor microenvironment or tumor-draining lymph nodes, or both, prior to or concurrent with the administration of the cancer vaccine. In another aspect, if the patient has an increase in the level or activity of T_{FR}, ST2 Treg, highly suppressive Treg, activated Treg, or effector Treg cells in a tumor microenvironment or tumor-draining lymph nodes or a high level or activity of T_{FR}, ST2 Treg, highly suppressive Treg, activated Treg, or effector Treg cells systemically, further comprising administering a T_{FR}, ST2 Treg, highly suppressive Treg, activated Treg, or effector Treg cell depleting therapy capable of transiently depleting T_{FR}, ST2 Treg, highly suppressive Treg, activated Treg, or effector Treg cells systemically prior to or concurrent with the administration of the cancer vaccine. In another aspect, if the patient has an increase in the level or activity of T_{FR}, ST2 Treg, highly suppressive Treg, activated Treg, or effector Treg cells in a tumor microenvironment or tumor-draining lymph nodes, further comprising administering a T_{FR}, ST2 Treg, highly suppressive Treg, activated Treg, or effector Treg cell depleting therapy capable of preferentially or transiently depleting T_{FR} cells in the tumor microenvironment or tumor-draining lymph nodes prior to or concurrent with the administration of the cancer vaccine. In another aspect, if the patient has a low level or decrease or low activity of T_{FR}, ST2 Treg, highly suppressive Treg, activated Treg, or effector Treg cells in a tumor microenvironment or tumor-draining lymph node, further comprising administering an immunotherapy agent or antibody. In another aspect, if the patient has a low level or decrease or low activity of T_{FR}, ST2 Treg, highly suppressive Treg, activated Treg, or effector Treg cells systemically, further comprising administering an immunotherapy agent or antibody. In another aspect, the T_{FR}, ST2 Treg, highly suppressive Treg, activated Treg, or effector Treg cell depleting therapy is provided in conjunction with, or followed by, the cancer vaccine. In another aspect, the method further comprises providing a cancer treatment selected from a checkpoint inhibitor or other immunotherapy agent or antibody. In another aspect, the checkpoint inhibitor or immunotherapy agent comprises an antibody directed against a cellular protein selected from at least one of: PD-1, PD-L1, CTLA-4, TIM-3, BTLA, LAG-3, or TIGIT. In another aspect, the immunotherapy agent comprises an antibody directed against a cellular protein selected from at least one of: PD-1, PD-L1, TIM-3, BTLA, LAG-3, or TIGIT. In another aspect, the T_{FR} cell depleting therapy comprises administering one or more of the following: a modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on T_{FR} cells when compared to T_{REG} cells and other T cell populations, wherein the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off. In another aspect, the T_{FR}, ST2 Treg, highly suppressive Treg, activated Treg, or effector Treg cell depleting therapy does not substantially reduce or eliminate T_{REGS}. In another aspect, the PI3K inhibitor is a selective Phosphoinositide 3-kinase δ inhibitor. In another aspect, the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114. In another aspect, the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺GITR⁺ T cells, or any combination thereof. In another aspect, the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1. In another aspect, the cancer is selected from a colorectal, a melanoma, a lung, a liver, a head and neck, and a breast cancer. In another aspect, the patient sample is a tumor biopsy. In another aspect, the step of determining the level or activity of T follicular regulatory (T_{FR}), ST2 Treg, highly suppressive Treg, activated Treg, or effector Treg cells in the sample is performed by measuring mRNA, protein, or both. In another aspect, the cancer vaccine is a tumor cell vaccine, an antigen vaccine, a dendritic cell vaccine, a DNA vaccine, an mRNA vaccine or a vector-based vaccine. In another aspect, the cancer vaccine is directed to a cancer antigen selected from at least one of: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, NY-ESO-1, LAGE-1, SSX-1, SSX-2 (HOM-MEL-40), SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RARalpha fusion protein, PTPRK, K-ras, N-ras, Triosephosphate isomerase, GnTV, Herv-K-mel, Lage-1, NA-88, /Lage-2, SP17, and TRP2-Int2, (MART-I), gp100, TRP-1, TRP-2, MAGE-1, MAGE-3, pi5(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, CDK4, Mum-1, p16, TAGE, PSMA, PSCA, CT7, telomerase, 43-9F, 5T4, 791Tgp72, alpha-fetoprotein, 13HCG, BCA225, BTAA, CA 125, CA 15-3, CA 195, CA 242, CA-50, CAM43, CD68\KP1, CO-029, FGF-5, G250, Ga733, HTgp-175, M344, MA-50, MG7-Ag, MOV18, NB\170K, NY-CO-1, RCAS1, SDCCAG16, TA-90, TAAL6, TAG72, TLP, TPS; or immunogenic fragments that comprise an epitope of any of the foregoing antigens.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B show: FIG. 1A, Luminex analysis of supernatants from an in vitro assay, depicted is the concentration of secreted interferon-g, Interleukin-2 and Tumor necrosis factor-a. FIG. 1B, fold-change reduction in secretion of indicated cytokines between T_{REG} and T_{FR} cells at a 4:1 ratio of CD8 T cells to either T_{REG} or T_{FR} cells. Data are mean+/−S.E.M.; All data are representative of two independent experiments.

FIGS. 2A to 2F show, FoxP3^{YFPcre/YFPcre} Bcl6^{fl/fl} (T_{FR} knockout) mice or FoXP3^{YFPcre/YFPcre} Bcl6^{+/+} control mice were s.c. inoculated with B16F10-OVA cells and treated with isotype control or anti-PD-1 Abs at indicated time points. Depicted are the tumor volume (FIG. 2B), the frequency of Gzmb⁺CD8⁺ T cells in tumor-draining lymph nodes (FIG. 2C, Col. 1 ctrl+isotype ctrl, Col. 2 ctrl+anti-PD-1, Col. 3 T_{FR} ko+isotype ctrl, Col. 4, T_{FR} ko+anti-PD-1), representative FACS plots (FIG. 2D, 2E) and the frequencies of dendritic cell populations (FIG. 2F) in tumor-draining lymph nodes of mice treated as indicated in (FIG. 2A), n=7-9 mice/group. Data are mean+/−S.E.M.; Significance for comparisons were computed using one-way ANOVA test comparing the mean of each column with the mean of the control column (ctrl+isotype ctrl). All data are representative of two independent experiments.

FIG. 3 shows the early and late assessment of TC1 mice treated with antibodies and a DNA vaccine (i.m.) at indicated time points.

FIGS. 4A to 4D show that PI3Kδ-inhibition drives anti-tumor immunity but causes significant irAEs. FIG. 4A, Swimlane plot depicting treatment regimen, intervals, and occurrence and grade of irAEs in PI3Kδi-treated (top plot) and placebo-treated patients (bottom plot), patients with PR or CR in PI3Kδi-treated patients are highlighted in magenta. FIG. 4B,4D, Volcano plots of whole tumor RNA-seq analysis (FIG. 4B) or bulk RNA-seq analysis of purified tumor-infiltrating CD8⁺ T cells (FIG. 4D) comparing AMG-319 to placebo treated patients. Differentially expressed genes between pre and post-treatment samples are highlighted and were called by DEseq2, adjusted p-values were calculated with the Benjamini-Hochberg method. Depicted are transcripts that change in expression more than 0.75-fold and adjusted P value of ≤0.1 (FIG. 4B) or <0.05 (FIG. 4D). FIG. 4C, Median cell count of FOXP3⁺ cells in pre- and post-treatment samples of placebo- or AMG319-treated patients. AMG-319-treated patients have been further stratified into patients where the interval between stopping of treatment and IHC assessment was >4 days (Long interval, LI) or <1 day (short interval, SI), P=0.015 for SI. Data are mean+/−S.E.M and statistical significance for comparisons was computed using two-tailed Wilcoxon matched-pairs signed rank test (FIG. 4C). Differential expression analysis (FIG. 4B, 4D) was performed using DESeq2 (v1.24.0).

FIGS. 5A to 5F show that PI3Kδ inhibition affects distinct T_{REG} cell subtypes. FIG. 5A,5B, Uniform manifold approximation and projection (UMAP) plots single-cell transcriptomes and TCR sequence data of FOXP3⁺CD4⁺ T cells in placebo-treated control mice (FIG. 5A, n=3 mice) and PI-3065-treated mice (FIG. 5B, n=3 mice); size scale indicated degree of clonal expansion. FIG. 5D, 5D, Violin plots show Seurat normalized expression levels of highlighted genes in the indicated clusters pertaining to FIG. 5A, 5B, the center line depicts the median, edges delineate the 25th and 75th percentiles and whiskers depict minimum and maximum values. FIG. 5E, Scatter plots showing Seurat normalized expression levels of highlighted genes in colonic T_{REG} cells in placebo-treated and PI-3065-treated mice, the dashed line indicates the expression cut-off. FIG. 5F, RNA velocity analysis visualized by UMAP, depicting likely developmental trajectories of T_{REG} cells pertaining to (FIG. 5A, 5B), arrows indicate velocity streamlines.

FIGS. 6A to 6D shows that PI3Kδ inhibition exacerbates colitis. FIG. 6A, mice were fed either a control diet or a diet containing the PI-3065 PI3Kδ inhibitor for the duration of the experiment and were additionally treated with 2.5% DSS from day 14-20. Depicted is the change in body weight when compared to body weight pre-treatment (day 0), n=10 mice/group, P<0.0001. FIG. 6B, shown are representative sections from H&E histology scans and colitis scoring from zinc-formalin fixed colonic tissue sections from placebo and PI3Kδ inhibitor-treated mice pertaining to (FIG. 6A), n=10 mice for placebo-treated mice and n=9 for PI3Kδ inhibitor-treated mice (one mouse died prior to experimental endpoint), P<0.0001 for inflammation, extent, crypt damage and overall colitis scoring, representative samples from the H&E staining are highlighted in magenta. FIG. 6C, Mice were inoculated s.c. with B16F10-OVA cells and fed either a control diet or a diet containing the PI-3065 PI3Kδ inhibitor (infrequent-dosing=PI3Kδi for 2 days followed by 5 days off drug; Intermittent-dosing=PI3Kδi for 4 days followed by 3 days off drug; Continuous-dosing=PI3Kδi for the duration of the experiment). Tumor volume (FIG. 6C) and flow-cytometric analyses of cell frequencies (FIG. 6D) of mice treated as indicated, n=6 mice for placebo, n=7 mice for intermittent-dosing, n=8 mice for continuous-dosing and infrequent-dosing; P=0.0023 for Control versus intermittent-dosing (FIG. 6C), P=0.0059 for Control versus continuous-dosing (c); P=0.003 for Control versus high-dosing and P=0.0003 for high-dosing versus intermediate dosing and low-dosing (left panel), P=0.0005 for Control versus high-dosing and P=0.0001 for high-dosing versus intermediate dosing and P<0.0001 for control versus low-dosing (left middle panel), P=0.0086 for Control versus high-dosing, P=0.045 for Control versus intermediate-dosing and P<0.0001 for high-dosing versus intermediate-dosing and low-dosing. Not significant, P=0.1234; *P=0.0332; ***P=0.0002; and ****P<0.0001. Data are mean+/−S.E.M and statistical significance for comparisons was computed using two-tailed Mann-Whitney test (FIG. 6A-6C) or one-way ANOVA comparing the mean of each group with the mean of each other group followed by Dunnett's test (FIG. 6D); data are representative of at least two independent experiments.

FIGS. 7A to 7F show that continuous dosing drives pathogenic T_{C}17 responses. Mice were inoculated s.c. with B16F10-OVA cells and fed either a control diet or a diet containing the PI-3065 PI3Kδ inhibitor, treatment conditions as in (FIGS. 6C, 6D). FIG. 7A, depicted is Seurat clustering visualized by UMAP of CD8⁺ T cells in colonic tissue at day 18 after tumor inoculation of mice treated as indicated, pie charts depict the percentage of each cluster in the different treatment conditions. FIG. 7B, Heatmap comparing gene expression of cells in all clusters. Depicted are transcripts that change in expression more than 0.5-fold and adjusted P value of <0.05, DEGs were called by MAST analysis, adjusted p-values were calculated with the Benjamini-Hochberg method. FIG. 7C, Seurat-normalized expression of Ifng (top left), Il17a (top right), Fcer1g (bottom left) and Mki67 (bottom right) in the different clusters. FIG. 7D, Clone size of cells in indicated clusters in UMAP space. FIG. 7E, Euler diagrams show the clonal overlap between the CD8⁺ T cells in the different clusters. FIG. 7F, RNA velocity analysis visualized by UMAP depicting likely developmental trajectories of CD8⁺ T cells, arrows indicate velocity streamlines.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The inventors have previously detected T-follicular Regulatory (TFRs) cells and explored their role in immune checkpoint therapies. The inventors now demonstrate that patients can be stratified into at least 2 groups: (1) patients with high levels and/or activity of TFR cells (intratumoral or systemically), in which case these patients benefit from targeted strategies to deplete intratumoral TFR cells or strategies that might deplete TFR cells systemically (but then only transiently); and (2) patients with low levels and/or activity of TFR cells (intratumoral or systemically), in which case these patients can go on to other immunotherapy treatments like anti-PD-1.

It has now been found that Phosphoinositide 3-kinase (PI3K) inhibitors, specifically Phosphoinositide 3-kinase δ inhibitors, are capable of broadly depleting TFRs in both the tumor microenvironment and systemically. Systemic depletion of TFRs can result in immune related toxicity and/or immune related adverse events (irAE's). The data demonstrate the cancer-immunotherapy potential of PI3Kδ inhibition in humans, but its modulation will need to be carefully balanced to harness its anti-tumor capacity while minimizing immune related toxicity caused by a systemic depletion of tissue-resident TFR cells. Further, patients should be screened/stratified based on the presence or level of intratumoral TFR cells and CD8⁺ T cells to identify patients who should receive Phosphoinositide 3-kinase δ inhibitors for cancer treatment.

As used herein, the terms “treatment”, “treating”, and the like, may include amelioration or elimination of a developed disease or condition once it has been established or alleviation of the characteristic symptoms of such disease or condition, specifically, the effects of follicular regulatory T cells (T_{FR}). For example, that patient may be treated for with a Phosphoinositide 3-kinase inhibitor for the treatment of cancer. One specific example is the use of Phosphoinositide 3-kinase δ specific inhibitors, which in addition to treating a cancer, eliminate TFRs, and thus increasing the likelihood of causing immune related toxicity and/or immune related adverse events (irAE's) from the inhibition or reduction of TFRs. Other examples include the treatment of a disease or condition that would benefit from preventing excessive activation or accumulation of PD-1 expressing follicular regulatory T cells (T_{FR}), e.g., anti-PD-1 treatment in cancer. The treatment would also target the indiscriminate depletion of FOXP3-expressing (T_{REG}+T_{FR}) cells, which results in immune related adverse events (irAEs). As used herein, these terms “treatment”, “treating”, and the like, may also encompass, depending on the condition of the subject, preventing the onset of a disease or condition or of symptoms associated with the disease or condition, including, for example, reducing the immune suppression caused by T_{FR} cells of cytotoxic tumor infiltrating lymphocytes (TIL). Such prevention or reduction prior to affliction may refer to administration of a therapeutic compound to a subject that is not at the time of administration afflicted with the disease or condition.

As used herein, the term “preventing” refers to preventing the indiscriminate depletion of FOXP3-expressing (T_{REG}+T_{FR}) cells, which might cause irAEs. Selectively depleting intratumoral TFR cells (without or with minimal depletion of T_{REG} cells) minimizes irAEs while maintaining treatment efficacy, especially in combination with anti-PD-1 therapy.

As used herein, these terms “subject” or “patient” can be any mammal, including a human and the treatment can be provided in vivo or cells can be treated in vitro and then returned to the subject or patient.

As used herein, the terms “standard control”, “control” or “control biological sample” refers to a sample, measurement, or value that serves as a reference, usually a known reference, for comparison to a subject biological sample, test sample, measurement, or value. For example, a test biological sample can be taken from a patient suspected of having a cancer or through the generation of a reference level for specific tumor types or immune related adverse events (irAEs). A standard control can represent an average measurement or value gathered from a T_{FR} population of similar individuals that do not have a given disease or condition (i.e., standard control population), e.g., healthy individuals with a similar medical background, same age, weight, etc. that do not have a cancer or immune related adverse events (irAEs). As shown herein, irAEs are caused by immunotherapy-mediated depletion (e.g., CTLA-4 antibodies) of suppressive FOXP3-expressing cells in multiple tissues (not only cancer tissue). Thus, specifically targeting and depleting intratumoral T_{FR} cells decreases irAEs while maintaining treatment efficacy. A standard control value can also be obtained from the same individual, e.g., from an earlier-obtained sample, prior to disease or condition (e.g., a cancer or immune related adverse events (irAEs)), or prior to treatment. One of skill in the art will understand which standard controls are valuable in a given situation and be able to analyze data based on comparisons to standard control values. Standard controls are also valuable for determining the significance of data. For example, if values for a given parameter are widely variant in standard controls, variation in test samples will not be considered as significant.

As used herein, the term “immune related adverse events (irAEs)” refers to immune-mediated toxicity or immunopathology in non-malignant organs (i.e., through the stimulation of CD4 or CD8 T cell proliferation or activity (i.e., cytotoxicity)).

As used herein, the term “T_{FR} cell population” refers to a cell population which has been processed so as to identify the cell population from other cell populations with which it is normally associated in its naturally occurring state using the various markers described herein, including both cell surface markers, but also the expression of genes or proteins that remain intracellularly and can be measured in vivo or ex vivo. The purified T_{FR} cell population can, thus, represent an enriched cell population in that the relative concentration of the cell population in a sample can be increased following such processing in comparison to its natural state. Alternatively, the T_{FR} cell population can be reduced by at least 50%, 60%, 70%, 80%, or at least 90%, or at least 95% or 100% in comparison to its natural state (i.e., pre-treatment) to prevent their immune suppressive activity. Such purified cell population may, thus, represent a cell preparation that can be further processed so as to obtain commercially viable preparations.

As used herein, the term “ST2 Treg (ST2 Treg)” refers to a CD4⁺ T cell that expresses ST2 (also known as IL1RL1), which is the functional receptor for interleukin (IL)-33 in stimulating regulatory T cell (Treg), Gata3, Klrg1, CD44, Interleukin 10, Id2, Areg, PD-1, Tgfb1, Ccr8, Icos, and Foxp3. ST2 Treg cells maintain self-tolerance and immune homeostasis and play critical roles in human diseases, such as autoimmune disease, allergy, and cancer. In the context of the present invention, the frequency of these cells is high in primary tumor or tumor-draining lymph node(s). If ST2 Treg cells are present in the primary tumor or tumor-draining lymph node(s), these cells are targeted with a Treg/Tfr cell targeting or depleting agent, as described herein, which can also be paired with a cancer vaccine.

As used herein, the terms “highly suppressive Treg”, “activated Treg (aTreg)”, and “effector Treg” refer to a highly suppressive, activated, and/or effector regulatory CD4⁺ T cell that expresses CD44, Foxp3, Cd25, Interleukin-10 and lack expression of CD62L and CD45RA. Like ST2 Treg, if Treg cells are present in the primary tumor or tumor-draining lymph node(s), these Treg cells are targeted with a Treg/Tfr cell targeting or depleting agent, as described herein, which can also be paired with a cancer vaccine.

Agents for reducing or eliminating T_{FR} cells may be processed so as to be part of a pharmaceutical composition, such as those taught herein. Non-limiting examples include anti-CTLA-4, anti-IL1R2, anti-4-1BB, anti-TNFR2, anti-ICOS, anti-GITR, anti-OX40, and/or anti-CCR8, that lead to T_{FR} depletion. For example, in one embodiment, the cell preparation can be prepared for transportation or storage in a serum-based solution containing necessary additives, which can then be stored or transported in a frozen form. In doing so, the person of skill will readily understand that the cell preparation is in a composition that includes a suitable carrier, which composition is significantly different from the natural occurring separate elements.

For example, the serum-based preparation may comprise human serum or fetal bovine serum, which is a structural form that is markedly different from the form of the naturally occurring elements of the preparation. The resulting preparation includes cells that are in dormant state, for example, that may have slowed-down or stopped intracellular metabolic reactions and/or that may have structural modifications to their cellular membranes. The resulting preparation includes cells that can, thus, be packaged or shipped while minimizing cell loss which would otherwise occur with the naturally occurring cells. This property of minimizing cell loss of the resulting preparation/composition is markedly different from properties of the cells by themselves in nature. A person skilled in the art would be able to determine a suitable preparation without departing from the present disclosure.

As used herein, the term “carrier” refers to any carrier, diluent or excipient that is compatible with the herein described composition that reduces or eliminates T_{FR}, such as, anti-CTLA-4, anti-IL1R2, anti-4-1BB, anti-ICOS, anti-GITR, anti-OX40, and/or anti-CCR8 (including antibodies) that cause TFR depletion or inactivation. Suitable acceptable carriers known in the art include, but are not limited to, water, saline, glucose, dextrose, buffered solutions, and the like. Such a carrier is advantageously non-toxic or has a limited effect on non-T_{FR} immune cells and not harmful to the subject. It may also be biodegradable. The carrier may be a solid or liquid acceptable carrier. A suitable solid acceptable carrier is a non-toxic carrier. For instance, this solid acceptable carrier may be a common solid micronized injectable such as the component of a typical injectable composition for example, but without being limited to, kaolin, talc, calcium carbonate, chitosan, starch, lactose, and the like. A suitable liquid acceptable carrier may be, for example, water, saline, DMSO, culture medium such as DMEM, and the like. The person skilled in the art will be able to determine a suitable acceptable carrier for a specific application without departing from the present disclosure.

As used herein, the terms “determining,” “measuring,” “evaluating,” “assessing,” and “assaying,” as used herein, generally refer to any form of measurement, and include determining if T_{FR} cells are present or not in a biological sample. In addition, these can also be used to determine the abundance of T_{FR} cells. These terms include both quantitative and/or qualitative determinations, which both require sample processing and transformation steps of the biological sample. Assessing may be relative or absolute. The phrase “assessing the presence of” can include determining the amount of something present, as well as determining whether it is present or absent.

As used herein, the term “therapeutically effective amount” may include the amount necessary to allow the component or composition that prevent the T_{FR} cells from performing their immunological role without causing overly negative effects in the host to which the component or composition is administered. In one example, the agents reduce or eliminate T_{FR} cells (or their activity), but do not significantly affect or deplete T_{REG} cells. The exact amount of the components to be used or the composition to be administered will vary according to factors such as the type of condition being treated, the type and age of the subject to be treated, the mode of administration, as well as the other ingredients in the composition.

As used herein, the term “expression” refers to a level of expression of a gene or a protein, that is transcribed from the gene. Generally, an “expression” level is determined by measuring the expression level of a gene of interest for a given cell population, determining the median expression level of that gene for the cell population, and comparing the expression level of the same gene for a particular cell to the median expression level for a different cell population. For example, if the expression level of a gene of interest for the single cell population is determined to be above the median expression level of the patient population, that cell is determined to have high expression of the gene of interest. Alternatively, if the expression level of a gene of interest for the cell population is determined to be below the median expression level of a normal cell population, that cell is determined to have low expression of the gene of interest.

As used herein when referring to a cell surface or other detectable marker, the terms “high” or “high expression” refers to a statistically significant increase in expression compared to naïve T cells. In certain embodiments, a statistically significant increase in expression refers to at least one log higher expression when compared to naïve T cells; in other cases, it can be 2 or even 3 logs higher. The expression can be measured with any number of methods, for example, fluorescence activated cell sorting, RNA-expression, luminescent or chemiluminescent platforms, Illumina®, MesoScale®, or other similar systems. As used herein, for example, when referring to the surface expression of PD-1^{high}, in the context of T cells (e.g., T_{FR}), are T cells that have a statistically significant increased expression when compared to naïve T cells. This statistically significant increase refers to, in certain embodiments, at least one log higher surface expression.

As used herein when referring to a level of activity, the terms “high” or “high activity” refers to a statistically significant increase in the effector activity of the T_{FR} cells compared to naïve T cells. In certain embodiments, a statistically significant increase in activity refers to at least one log higher activity when compared to naïve T cells; in other cases, it can be 2 or even 3 logs higher. The activity can be measured with any number of methods, for example, changes in the expression of certain genes or changes in the release of certain cytokines, which can be measured by RNA-expression, luminescent or chemiluminescent platforms, Illumina®, MesoScale®, or other similar systems. As used herein, for example, when referring to changes in the expression of certain genes or changes in the release of certain cytokines, in the context of T cells (e.g., T_{FR}), are T cells that have a statistically significant increase in the expression of certain genes or release of certain cytokines, when compared to naïve T cells. This statistically significant increase refers to, in certain embodiments, at least one log higher activity.

As used herein, the terms “cancer vaccine” or “cancer immunization” refer to a composition capable of inducing active immunity against at least one cancer antigen. Immunization with a cancer vaccine or immunization can result in, e.g., production of antibodies against a cancer antigen, such as an antigen on a cancerous cell, the activation of certain cells, in particular antigen-presenting cells, T lymphocytes (in particular T-CD8+ cells), Natural Killer cells, and B lymphocytes. A cancer vaccine can be a composition for prophylactic purposes or for therapeutic purposes or both.

As used herein the term “antigen” refers to a molecule capable of being specifically bound by an antibody or presented to a T cell receptor (TCR) if processed and presented by a major histocompatibility (MHC) molecule, or its equivalent. As such, the term “antigen” also encompasses T-cell epitopes. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes).

As used herein, the term “cancer antigen” refers to an antigen that is found in, or on, or is characteristic of, a tumor tissue. There are multiple types of cancer vaccines. Non-limiting examples of cancer vaccines include tumor cell vaccines, DNA vaccines, antigen vaccines, dendritic cell vaccines, and vector-based vaccines.

Non-limiting examples of cancer antigens include, e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, NY-ESO-1, LAGE-1, SSX-1, SSX-2 (HOM-MEL-40), SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-All, hsp70-2, KIAAO205, Mart2, Mum-2, and 3, neo-PAP, myosin class I, OS-9, pml-RARalpha fusion protein, PTPRK, K-ras, N-ras, Triosephosphate isomerase, GnTV, Herv-K-mel, Lage-1, NA-88, /Lage-2, SP17, and TRP2-Int2, (MART-I), gp100, TRP-1, TRP-2, MAGE-1, MAGE-3, p15(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, pi80erbB-3, c-met, nm-23H1, PSA, TAG-72-4, CA 19-9, CA 72-4, CAM 17.1, NuMa, K-ras, CDK4, Mum-1, p16, TAGE, PSMA, PSCA, CT7, telomerase, 43-9F, 5T4, 791Tgp72, alpha-fetoprotein, 13HCG, BCA225, BTAA, CA 125, CA 15-3, CA 195, CA 242, CA-50, CAM43, CD68\KP1, CO-029, FGF-5, G250, Ga733, HTgp-175, M344, MA-50, MG7-Ag, MOV18, NB\170K, NY-CO-1, RCAS1, SDCCAG16, TA-90, TAAL6, TAG72, TLP, TPS, CD19, CD20, CD21, CD23, CD22, CD33 CD35, CD44, CD45, CD46, CD5, CD52, CD55, CD59, CDC27; or immunogenic fragments that comprise an epitope of any of the foregoing antigens.

As used herein, the term “cancer” refers to both solid tumors and blood-borne tumors. The term cancer includes diseases of the skin, tissues, organs, bone, cartilage, blood and vessels. The term “cancer” includes primary and/or metastatic cancers. Examples of cancers that may be treated by methods and compositions of the present invention include, e.g., cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, e.g., acinar cell carcinoma; adenocarcinoma; adenocarcinoma; adenocarcinoma in adenomatous polyp; adenocarcinoma w/squamous metaplasia; adenocarcinoma, familial polyposis coli; adenoid cystic carcinoma; adenosquamous carcinoma; adrenal cortical carcinoma; alveolar rhabdomyosarcoma; amelanotic melanoma; ameloblastic fibrosarcoma; ameloblastic odontosarcoma; ameloblastoma, malignant; apocrine adenocarcinoma; astroblastoma; astrocytoma; basal cell carcinoma; basophil carcinoma; basophilic leukemia; branchiolo-alveolar adenocarcinoma; brenner tumor; carcinoid tumor, malignant; carcinoma; carcinoma, undifferentiated; carcinosarcoma; cerebellar sarcoma; ceruminous; cholangiocarcinoma; chondroblastoma; chondrosarcoma; chordoma; choriocarcinoma; chromophobe carcinoma; clear cell adenocarcinoma; combined hepatocellular carcinoma and cholangiocarcinoma; cystadenocarcinoma; dysgerminoma; embryonal carcinoma; embryonal rhabdomyosarcoma; endometroid carcinoma; eosinophilic leukemia; ependymoma; epithelioid cell melanoma; erythroleukemia; Ewing's sarcoma; extra-mammary paraganglioma; fibrillary astrocytoma; fibrosarcoma; fibrous histiocytoma; follicular adenocarcinoma; ganglioneuroblastoma; gastrinoma, malignant; giant and spindle cell carcinoma; giant cell tumor of bone; glioblastoma; glioma, malignant; glomangiosarcoma; granular cell carcinoma; granular cell tumor; granulosa cell tumor, malignant; hairy cell leukemia; hemangioendothelioma; hemangiopericytoma; hemangiosarcoma; hepatoblastoma; hepatocellular carcinoma; Hodgkin's disease; Hodgkin's lymphoma; immunoproliferative small intestinal disease; infiltrating duct carcinoma; inflammatory carcinoma; juxtacortical osteosarcoma; kaposi's sarcoma; leiomyosarcoma; leukemia; Leydig cell tumor, malignant; lipid cell tumor, malignant; liposarcoma; lobular carcinoma; lymphangiosarcoma; lymphoepithelial carcinoma; lymphoid leukemia; lymphosarcoma cell leukemia; malignant histiocytosis; malignant lymphoma; malignant lymphoma, follicular; malignant lymphoma, large cell, diffuse; malignant lymphoma, small lymphocytic; malignant melanoma; malignant melanoma in giant pigmented nevus; mast cell leukemia; mast cell sarcoma; medullary carcinoma; megakaryoblastic leukemia; meningioma; mesenchymal chondrosarcoma; mesenchymoma; mesonephroma; mesothelioma; mixed tumor; monocytic leukemia; mucinous adenocarcinoma; mucinous cystadenocarcinoma; mucoepidermoid carcinoma; mullerian mixed tumor; multiple myeloma; mycosis fungoides; myeloid leukemia; myeloid sarcoma; myxosarcoma; neoplasms; nephroblastoma; neurilemmoma; neuroblastoma; neurofibrosarcoma; non-Hodgkin's lymphomas; nonencapsulating sclerosing carcinoma; odontogenic tumor; olfactory neurogenic tumor; oligodendroblastoma; oligodendroglioma; osteosarcoma; ovarian stromal tumor; oxyphilic adenocarcinoma; Paget's disease; papillary adenocarcinoma; papillary and follicular adenocarcinoma; papillary carcinoma; papillary cystadenocarcinoma; papillary serous cystadenocarcinoma; papillary transitional cell carcinoma; paraganglioma, malignant; paragranuloma; pheochromocytoma; phyllodes tumor; pilomatrix carcinoma; pinealoma; plasma cell leukemia; primitive neuroectodermal; protoplasmic astrocytoma; retinoblastoma; rhabdomyosarcoma; roblastoma; sarcoma; sebaceous adenocarcinoma; Sertoli cell carcinoma; signet ring cell carcinoma; skin appendage carcinoma; small cell carcinoma; solid carcinoma; squamous cell carcinoma; stromal sarcoma; superficial spreading melanoma; synovial sarcoma; teratoma; thecoma, malignant; thymoma, malignant; trabecular adenocarcinoma; transitional cell carcinoma.

As used herein, the terms “treatment”, “treating”, and the like, may include amelioration or elimination of a developed disease or condition once it has been established or alleviation of the characteristic symptoms of such disease or condition, specifically, the effects of follicular regulatory T cells (T_{FR}). For example, that patient may be treated for with a Phosphoinositide 3-kinase inhibitor for the treatment of cancer. One specific example is the use of Phosphoinositide 3-kinase δ specific inhibitors, which in addition to treating a cancer, eliminate TFRs, and thus increasing the likelihood of causing immune related toxicity and/or immune related adverse events (irAE's) from the inhibition or reduction of TFRs. Other examples include the treatment of a disease or condition that would benefit from preventing excessive activation or accumulation of PD-1 expressing follicular regulatory T cells (T_{FR}), e.g., anti-PD-1 treatment in cancer. The treatment would also target the indiscriminate depletion of FOXP3-expressing (T_{REG}+T_{FR}) cells, which results in immune related adverse events (irAEs). As used herein, these terms “treatment”, “treating”, and the like, may also encompass, depending on the condition of the subject, preventing the onset of a disease or condition or of symptoms associated with the disease or condition, including, for example, reducing the immune suppression caused by T_{FR} cells of cytotoxic tumor infiltrating lymphocytes (TIL). Such prevention or reduction prior to affliction may refer to administration of a therapeutic compound to a subject that is not at the time of administration afflicted with the disease or condition.

As used herein, the term “preventing” refers to preventing the indiscriminate depletion of FOXP3-expressing (T_{REG}+T_{FR}) cells, which might cause irAEs. Selectively depleting intratumoral TFR cells (without or with minimal depletion of T_{REG} cells) minimizes irAEs while maintaining treatment efficacy, especially in combination with anti-PD-1 therapy.

As used herein, these terms “subject” or “patient” can be any mammal, including a human and the treatment can be provided in vivo or cells can be treated in vitro and then returned to the subject or patient.

As used herein, the terms “reference sample”, “standard control”, “control” or “control biological sample” refers to a sample, measurement, or value that serves as a reference, usually a known reference, for comparison to a subject biological sample, test sample, measurement, or value. For example, a reference level of T_{FR} cells can be determined for a known human population that does not have a cancer. The reference sample can be from the cancer patient, but obtained from another location, e.g., from the same organ but from an area that does not have a cancer, or from another organ. A reference sample is often a biological or patient sample, but can also include a collection of data, a reference database, or a value (such as the biological activity of the T_{FR} cells as described herein or the number of T_{FR} cells that is found in a population of individuals that do not have a cancer). For example, the reference sample might be drawn from an organ that contains few or no T_{FR} cells in steady state from the patient. A standard control can represent an average measurement or value gathered from a T_{FR} population of similar individuals that do not have a given disease or condition (i.e., standard control population), e.g., healthy individuals with a similar medical background, same age, weight, etc. that do not have a cancer or immune related adverse events (irAEs). As shown herein, irAEs are caused by immunotherapy-mediated depletion (e.g., CTLA-4 antibodies) of suppressive FOXP3-expressing cells in multiple tissues (not only cancer tissue). Thus, specifically targeting and depleting intratumoral T_{FR} cells decreases irAEs while maintaining treatment efficacy. A standard control value can also be obtained from the same individual, e.g., from an earlier-obtained sample, prior to disease or condition (e.g., a cancer or immune related adverse events (irAEs)), or prior to treatment. One of skill in the art will understand which standard controls are valuable in a given situation and be able to analyze data based on comparisons to standard control values. Standard controls are also valuable for determining the significance of data. For example, if values for a given parameter are widely variant in standard controls, variation in test samples will not be considered as significant.

As used herein, the term “immune related adverse events (irAEs)” refers to immune-mediated toxicity or immunopathology in non-malignant organs (i.e., through the stimulation of CD4 or CD8 T cell proliferation or activity (i.e., cytotoxicity)).

Agents for reducing, eliminating, or temporarily reducing T_{FR} cells may be processed so as to be part of a pharmaceutical composition, such as those taught herein. Non-limiting examples include anti-CTLA-4, anti-IL1R2, anti-4-1BB, anti-TNFR2, anti-ICOS, anti-GITR, anti-OX40, and/or anti-CCR8, that lead to T_{FR} depletion. For example, in one embodiment, the cell preparation can be prepared for transportation or storage in a serum-based solution containing necessary additives, which can then be stored or transported in a frozen form. In doing so, the person of skill will readily understand that the cell preparation is in a composition that includes a suitable carrier, which composition is significantly different from the natural occurring separate elements.

For example, the serum-based preparation may comprise human serum or fetal bovine serum, which is a structural form that is markedly different from the form of the naturally occurring elements of the preparation. The resulting preparation includes cells that are in dormant state, for example, that may have slowed-down or stopped intracellular metabolic reactions and/or that may have structural modifications to their cellular membranes. The resulting preparation includes cells that can, thus, be packaged or shipped while minimizing cell loss which would otherwise occur with the naturally occurring cells. This property of minimizing cell loss of the resulting preparation/composition is markedly different from properties of the cells by themselves in nature. A person skilled in the art would be able to determine a suitable preparation without departing from the present disclosure.

### Example 1. T_{FR} Cells Inhibit Anti-Tumor Immunity and are Responsive to Immune Checkpoint Blockade

An increased density of regulatory T cells (T_{REG}) in tumors has been linked to poor survival outcomes. In non-cancer settings, T_{REG} cells have been shown to differentiate into PD-1 expressing follicular regulatory T cells (T_{FR}) that restrain germinal center responses. It is not known whether such differentiation also occurs in the tumor microenvironment, and if so, whether such tumor-infiltrating TFR cells are molecularly distinct from T_{REG} cells or are activated by anti-PD1 therapy. In this example, the inventors show that T_{FR} cells are present in high numbers in human and murine tumor tissues, share T cell receptor (TCR) clonotypes with intratumoral T_{REG} cells and express high levels of PD-1. Single-cell TCR data, trajectory analyses and adoptive transfer studies indicate intratumoral conversion of T_{REG} to T_{FR} cells. When compared to T_{REG} cells, T_{FR} cells exhibited enhanced suppressive capacity in vitro and in vivo and expressed higher levels of molecules known to be linked to co-stimulation (4-1BB, ICOS, GITR), cell proliferation (Ki67), suppressive function (CTLA-4), and self-renewal potential (TCF-1), all features suggestive of superior functional properties.

In syngeneic tumor models, anti-PD-1 treatment increased the number of tumor-infiltrating TFR cells. Conditional knockout of T_{FR} cells or depletion of T_{FR} cells with anti-CTLA-4 antibody prior to anti-PD1 treatment, improved tumor control in mice. Notably, in a cohort of 271 melanoma patients, treatment with anti-CTLA-4 followed by anti-PD-1 at progression was associated with better long-term survival outcomes than anti-PD-1 or anti-CTLA-4 monotherapy, anti-PD-1 followed by anti CTLA-4 at progression or concomitant combination therapy. These findings illustrate that anti-PD1 therapy has the potential to regulate abundance and/or functionality of T_{FR} cells, and can thus induce a profoundly immunosuppressive milieu impeding anti-tumor immunity. Thus, indiscriminate use of anti-PD-1 therapy can prove detrimental in some patients.

Follicular regulatory T cells (T_{FR}) inhibit T and B cell responses to mitigate germinal center reactions in secondary lymphoid organs, impede humoral immunity towards self-antigens and display heightened suppressive capacity when compared to T_{REG} cells. T_{FR} cells are being characterized by their joint expression of the surface molecules CXCR5 and GITR, or by their co-expression of the transcription factors FOXP3 and BCL-6. Several studies have demonstrated that, depending on disease context and organ, cells of the T follicular lineage express varying levels of CXCR5 and BCL-6. Moreover, it has been shown that deletion of CXCR5 expression in FOXP3-expressing cells does not abrogate the development and maintenance of BCL-6+T_{FR} cells, indicating that distinct subsets of TFR cell cells exist, which not only differ in their expression of CXCR5 and BCL-6, but also in their expression of CD25.

Most tumors contain tertiary lymphoid structures and because cancerous cells frequently express self or altered-self antigens, the inventors investigated if T_{REG} and T_{FR} cells accumulate in parallel in the tumor microenvironment (TME) as a means of effective immune evasion. Non-limiting examples of cancers for which the stratification of TFRs, and treated based on the number and/or activity of TFRs using the present invention, can selected from, e.g., colorectal, melanoma, lung, liver, head and neck, and breast cancer.

The present inventors have previously found that TFRs account for a substantial proportion of tumor-infiltrating CD4⁺ T cells, and importantly, are highly responsive to immune checkpoint blockade. TFR cells play a pivotal role in anti-tumor immunity and in determining cancer immunotherapy treatment efficacy, including checkpoint blockade therapies. Certain cancer immunotherapies can be improved by depleting TFR cells in the tumor microenvironment (e.g., with anti-IL1R2 antibodies) prior to initiating treatment with anti-PD-1 or another immunotherapy.

The present inventors have now found that the systemic depletion of TFRs by Phosphoinositide 3-kinase δ inhibitors can result in irAE's. Therefore, PI3Kδ inhibitors need to be titrated carefully to more selectively deplete intratumoral TFR cells without substantially depleting TFR cells in other organs. Further, PI3Kδi might be especially useful in patients with high levels of intratumoral T_{REG} cells and an unfavorable ratio of T_{FR} versus CD8⁺ TILs in pre-treatment samples. Therefore, patients can be stratified for treatment with Phosphoinositide 3-kinase δ inhibitors based on the presence or level of TFR cells and CD8⁺ TILs prior to administration. The presence, absence, level or activation status of the patient's TFRs might be further indicative of whether a patient will experience adverse events from treatment with Phosphoinositide 3-kinase δ inhibitors and therefore can be used to identify patient who should or should not be administered Phosphoinositide 3-kinase δ inhibitors.

The present invention includes diagnostic and therapeutic uses that include stratifying patient populations as determined by the presence, absence, levels or activation status of TFRs, determining which patient population would be negatively impacted by the administration of a Phosphoinositide 3-kinase δ inhibitors, and then modifying the treatment dosing (amount per dose) or regimen (frequency of doses). A modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on T_{FR} cells when compared to T_{REG} cells and other T cell populations includes the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off.

The inventors further examine how adjustments to the dosage amount or schedule of Phosphoinositide 3-kinase δ inhibitors can provide depletion of TFRs more specifically in the tumor microenvironment, as opposed to in other tissues. By optimizing the dosage and regimen, it is possible to increase the likelihood of success of that cancer immunotherapy and/or reduce the likelihood or severity of immune related adverse events resulting from treatment. This is true for both the use of Phosphoinositide 3-kinase inhibitors (e.g., Phosphoinositide 3-kinase 6) as cancer treatments, but also for their use in selectively depleting TFR cells in the tumor microenvironment or tumor-draining lymph nodes, or transiently depleting TFR cells systemically, in combination with additional cancer therapies or treatments, for example, immune checkpoint inhibitors or immunotherapy antibodies.

Mouse experiments are conducted to ascertain whether Phosphoinositide 3-kinase δ inhibitors selectively deplete TFR cells in different organs, i.e., colonic tissue, as colitis is one of the major irAEs in patients receiving Phosphoinositide 3-kinase δ inhibitors. The inventors explore whether Phosphoinositide 3-kinase δ inhibitors can cause colitis or colonic tissue inflammation and whether Phosphoinositide 3-kinase δ inhibitors can be titrated (concentration, treatment regimen or both) to more selectively target intratumoral TFR cells, without substantially depleting TFR cells in other organs. It is also possible to demonstrate that Phosphoinositide 3-kinase δ inhibitors act by activating the immune system (i.e., by depleting TFR cells), instead of acting intrinsically on cancerous cells.

Moreover, the present invention can be used to build a repository for the levels of intra-tumoral T cell populations (i.e., CD8+ T cells and TFR cells) for different cancer types (melanoma, head and neck cancer, lung cancer, breast, colorectal, liver, etc.). The levels identified in the repositories make it possible to set thresholds for these intra-tumoral immune subsets for patient stratification. Further, these thresholds can also be used in randomized clinical trials.

### Example 2. Stratification of Patients

The inventors have previously detected T-follicular Regulatory (TFRs) cells and explored their role in immune checkpoint therapies. The inventors now demonstrate that patients can be stratified into at least 2 groups: (1) patients with high levels and/or activity of TFR cells (intratumoral or systemically), in which case these patients benefit from targeted strategies to deplete intratumoral TFR cells or strategies that might deplete TFR cells systemically (but then only transiently); and (2) patients with low levels and/or activity of TFR cells (intratumoral or systemically), in which case these patients can go on to other immunotherapy treatments like anti-PD-1.

The inventors further recognized that depleting TFR cells or FOXP3-expressing cells in general, or blocking their activity, even if systemically, enhances the efficacy of cancer vaccines. The data further demonstrate that a transient depletion of TFR cells or FOXP3-expressing cells, i.e., by short-term or intermittent treatment with PI3Kδ inhibitors or (ADCC-optimized) depleting antibodies including, but not limited to, anti-CTLA-4, anti-CCR8 or anti-IL1R2, or blocking the activity of these cells, enhances the efficacy of cancer vaccines while limiting the probability of irAEs. The data further show that this effect is mediated by an altered ratio of CD4+ T cells to TFR cells or more broadly of CD4+ T cells to FOXP3+ cells, leading to alterations in the frequency and/or activity of classical dendritic cells type 1 (cDC1) cells, which cross-present antigen (i.e. tumor associated antigens, tumor neoantigens, or any antigen in a given cancer vaccine) to CD8+ T cells. Thus, depletion of TFR cells or FOXP3-expressing cells or blockade of their functionality will improve cancer vaccine efficacy by altering the priming of tumor-associated antigen or tumor neoantigen specific CD8+ T cells and thus enhance anti-tumor immunity. The results further demonstrate that the ratio of CD4+ T cells to TFR cells and/or more broadly of CD4+ T cells to FOXP3+ T cells pre-treatment (i.e., immunotherapy or cancer vaccine) can be used for patient stratification to improve the likelihood of success of a given cancer immunotherapy or cancer vaccine. This patient stratification can be used either alone or alongside other established methods of patient stratification like tumor mutational burden or MHC intratumoral heterogeneity.

Follicular regulatory T cells (T_{FR}) inhibit T and B cell responses to mitigate germinal center reactions in secondary lymphoid organs, impede humoral immunity towards self-antigens and display heightened suppressive capacity when compared to T_{REG} cells. T_{FR} cells are being characterized by their joint expression of the surface molecules CXCR5 and GITR, or by their co-expression of the transcription factors FOXP3 and BCL-6. Several studies have demonstrated that, depending on disease context and organ, cells of the T follicular lineage express varying levels of CXCR5 and BCL-6. Moreover, it has been shown that deletion of CXCR5 expression in FOXP3-expressing cells does not abrogate the development and maintenance of BCL-6+T_{FR} cells, indicating that distinct subsets of TFR cell cells exist, which not only differ in their expression of CXCR5 and BCL-6, but also in their expression of CD25.

Most tumors contain tertiary lymphoid structures and because cancerous cells frequently express self or altered-self antigens, the inventors investigated if T_{REG} and T_{FR} cells accumulate in parallel in the tumor microenvironment (TME) as a means of effective immune evasion.

The present inventors have previously found that TFRs account for a substantial proportion of tumor-infiltrating CD4⁺ T cells, and importantly, are highly responsive to immune checkpoint blockade. T_{FR} cells play a pivotal role in anti-tumor immunity and in determining cancer immunotherapy treatment efficacy, including checkpoint blockade therapies. Certain cancer immunotherapies can be improved by depleting T_{FR} cells in the tumor microenvironment (e.g., with anti-IL1R2 antibodies) prior to an immunotherapy, such as a cancer vaccine therapy.

For example, adjustments to the dosage amount or schedule of Phosphoinositide 3-kinase δ inhibitors can provide depletion of TFRs more specifically in the tumor microenvironment, as opposed to in other tissues. By optimizing the dosage and regimen, it is possible to increase the likelihood of success of that cancer immunotherapy and/or reduce the likelihood or severity of immune related adverse events resulting from treatment. This is true for both the use of Phosphoinositide 3-kinase inhibitors (e.g., Phosphoinositide 3-kinase δ) as cancer treatments, but also for their use in selectively depleting TFR cells in the tumor microenvironment or tumor-draining lymph nodes, or transiently depleting T_{FR} cells systemically, in combination with additional cancer therapies or treatments, for example, immune checkpoint inhibitors or immunotherapy antibodies.

Moreover, the present invention can be used to build a repository for the levels of intra-tumoral T cell populations (i.e., CD8+ T cells and TFR cells) for different cancer types (melanoma, head and neck cancer, lung cancer, breast, colorectal, liver, etc.). The levels identified in the repositories make it possible to set thresholds for these intra-tumoral immune subsets for patient stratification. Further, these thresholds can also be used in randomized clinical trials.

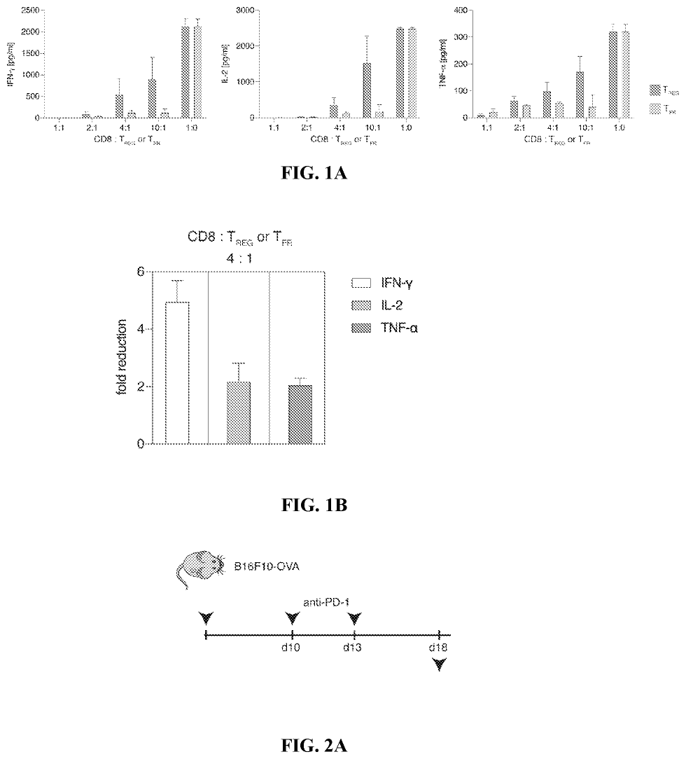

To experimentally validate that T_{FR} cells are more suppressive than T_{REG} cells, the inventors performed functional assays in vitro and in vivo. Surprisingly, it was found that T_{FR} cells inhibited CD8⁺ T cell proliferation more efficiently than T_{REG} cells, and also reduced their secretion of effector molecules interferon-γ, Interleukin (IL)-2 and Tumor necrosis factor-α (TNF-α) more effectively (FIG. 1A). Notably, when compared to T_{REG} cells, T_{FR} cells reduced the secretion of interferon-γ by CD8⁺ T cells ˜4-fold, and the secretion of IL-2 and TNF-α by CD8⁺ T cells ˜2-fold (FIG. 1B). These data demonstrate that T_{FR} cells are highly suppressive and show that they are able to actively diminish the effector functions of CD8⁺ T cells, even at low cell numbers.

FIGS. 1A and 1B show: FIG. 1A, Luminex analysis of supernatants from an in vitro assay, depicted is the concentration of secreted interferon-g, Interleukin-2 and Tumor necrosis factor-a. FIG. 1B, fold-change reduction in secretion of indicated cytokines between T_{REG} and T_{FR} cells at a 4:1 ratio of CD8 T cells to either T_{REG} or T_{FR} cells. Data are mean+/−S.E.M.; All data are representative of two independent experiments.

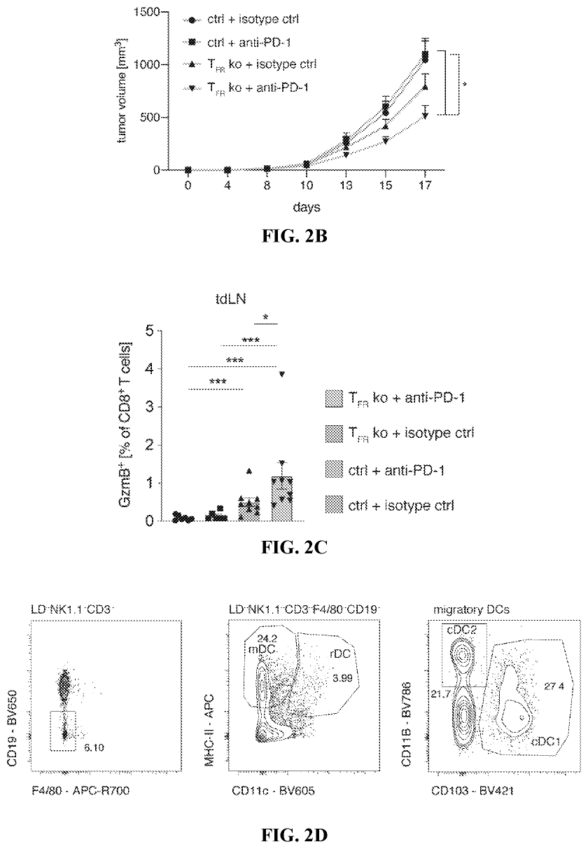

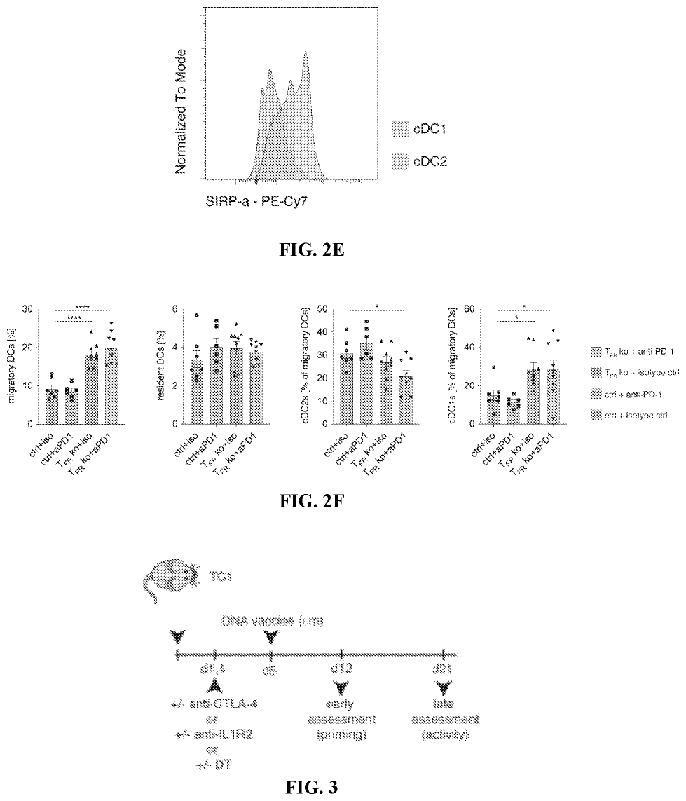

To assess the functional importance of T_{FR} cells in tumor development and to further corroborate that T_{FR} cells impair anti-PD-1 treatment efficacy, T_{FR} cell-deficient FoxP3^{YFP-crc}×Bcl6^{fl/fl} mice were used and assessed the tumor-draining axillary and inguinal lymph nodes in the B16F10-OVA model. It is demonstrated herein that T_{FR} cells can inhibit CD8⁺ T cell activity and cytokine secretion (FIGS. 1 A, 1B). It was found that T_{FR} cell deficiency results in increased granzyme B expression in CD8⁺ T cells (FIG. 2C). Crucially, a significant increase in the frequency of migratory dendritic cells (FIG. 2F) was also found. This increase was mediated by an elevated abundance of classical dendritic type I (cDC1) cells (FIG. 2F), which have been shown to efficiently cross-present antigens to CD8⁺ T cells. Together, these data indicate that T_{FR} cells regulate the priming and/or activity of CD8⁺ T cell activity in tumor-draining lymph nodes and indicate that a depletion of T_{FR} cells or blocking of their activity might improve the efficacy of vaccines, specifically, cancer vaccines.

FIGS. 2A to 2F show, FoxP3^{YFPcre/YFPcre} Bcl6^{fl/fl} (T_{FR} knockout) mice or FoxP3^{YFPcre/YFPcre} Bcl6^{+/+} control mice were s.c. inoculated with B16F10-OVA cells and treated with isotype control or anti-PD-1 Abs at indicated time points. Depicted are the tumor volume (FIG. 2B), the frequency of Gzmb⁺CD8⁺ T cells in tumor-draining lymph nodes (FIG. 2C), representative FACS plots (FIGS. 2D, 2E) and the frequencies of dendritic cell populations (f) in tumor-draining lymph nodes of mice treated as indicated in (FIG. 2A), n=7-9 mice/group. Data are mean+/−S.E.M.; Significance for comparisons were computed using one-way ANOVA test comparing the mean of each column with the mean of the control column (ctrl+isotype ctrl). All data are representative of two independent experiments.

T follicular regulatory (TFR or T_{FR}) cells and cancer vaccines. The inventors have shown that T_{FR} were prevalent in human and murine tumor tissues of several cancer types. Among tumor-infiltrating T cells, the inventors found that T_{FR} cells expressed the highest levels of CTLA-4 and PD-1, which renders them highly responsive to Immune check point blockade (ICB) therapies targeting these molecules. The inventors found that by increasing the abundance of T_{FR} cells, anti-PD-1 therapy can dampen anti-tumor immunity. Accordingly, in murine tumor models, the inventors show that depleting T_{FR} cells using either genetic (conditional knockout of T_{FR} cells) or pharmacological (anti-CTLA-4 antibody) approaches prior to anti-PD-1 treatment improved tumor control. In a large cohort of melanoma patients, the inventors found that sequential ICB (anti-CTLA-4 prior to anti-PD-1) was associated with better long-term survival outcomes when compared to concomitant combination therapy or monotherapy with either agent, highlighting the benefit of depleting T_{FR} cells to render tumors responsive to anti-PD1 therapy.

Targeting T_{FR} cells to improve efficacy of cancer vaccines. Vaccines targeting tumor-associated antigens (TAA) can enhance tumor control by eliciting CD4+ and CD8+ T cell responses, which can be further enhanced by ICB. However, similar to ICB, it remains unclear why cancer vaccines only work in a minority of patients. Conventional Type 1 Dendritic Cells (cDC1) have been established as a critical link that facilitates an effective priming of CD8+ T cells via antigen cross-presentation, a process that is licensed by CD4+ T cells6. The inventors found that T_{FR} cells reduce the abundance of cDC1 cells in tumor-draining lymph nodes, showing that T_{FR} cells are not only a key cellular target for ICB, but also a critical determinant in the priming of anti-tumor CD8+ T cell responses. Therefore, the inventors expect that reducing T_{FR} cell frequency will improve the efficacy of cancer vaccines by enhancing priming of tumor-specific CD8+ T cells.

IL1R2 is a novel immunotherapy target to selectively deplete T_{FR} cells. Although combination immune checkpoint blockade (ICB) (e.g., anti-CTLA-4+anti-PD-1) results in significantly higher overall response rates compared to monotherapy with either agent, it causes more frequent and severe immune-related adverse events (irAEs), thus limiting its use. Hence, translating the benefits of ICB to a broader patient cohort requires identifying and developing novel immunotherapy targets that combine high efficacy rates with low immune-related toxicity. The inventors have demonstrated that one such promising approach is selective depletion of T_{FR} cells (instead of targeting all FOXP3-expressing cells) by targeting a novel decoy receptor IL1R2, a surface molecule that unlike other T_{REG}-targeting molecules like CD25, CTLA-4 or CCR8, is more specifically expressed by T_{FR} cells in humans and mice.

Determine the mechanisms of T_{FR} cell-mediated suppression of anti-tumor immune responses. (A) Natural and ICB-induced anti-tumor responses: Genetic models can be used (T_{FR}-deficient mice; Foxp3^{YFP-cre}×Bcl6^{fl/fl}) to determine whether depletion of T_{FR} cells improves natural and anti-PD1-mediated anti-tumor immunity by altering the capacity of cDC1 to effectively prime tumor-specific CD8+ T cell responses. Having established that mice that are selectively deficient in T_{FR} cells display augmented anti-tumor immunity, it is possible to determine whether T_{FR} cell-deficiency augments the activity of tumor-antigen specific cytotoxic T cells and whether T_{FR} cells induce functional changes in cytotoxic T lymphocytes (CTLs) and other tumor-infiltrating lymphocytes. (B) Vaccine-induced anti-tumor responses: Cancer vaccines work by enhancing tumor-antigen cross-presentation via cDC1s. To investigate whether T_{FR} cell-deficiency improves the efficacy of cancer vaccines by modulating cDC1 function, it is possible to use a vaccine-responsive tumor-model that uses HPV-16 E7 antigen-expressing TC-1 tumor cells. In this model, a DNA vaccine encoding a long peptide sequence from within the E7 protein induces HPV E7-specific CD8+ T cell responses that rely on CD4+ T cell help, which in turn results in tumor control.

Develop Selective T_{FR} cell Depletion Strategies.
(A) IL1R2 protein expression studies. To evaluate the potential efficacy of selective T_{FR} depletion therapy versus other TREG-targeting immunotherapy drugs (i.e., CTLA-4 and CCR8) while taking into consideration their capacity to cause irAEs in a clinical setting, the expression patterns of these molecules can be assessed in immune cell types from multiple cancer types and non-malignant tissues.
(B) Selective depletion of T_{FR} cells: To translate the results into a therapy, it is possible to generate and assess the efficacy of T_{FR} cell-depleting antibodies by targeting IL1R2, a surface molecule identified to be highly expressed by T_{FR} cells. Given that anti-CTLA-4 antibodies non-specifically target both TREG and T_{FR} cells, potentially causing irAEs in non-malignant tissues, it is now possible to assess whether targeting IL1R2, recapitulates the anti-tumor effects of T_{FR} cell-deficient mice. A Beacon platform can be used to generate multiple potential antibody clones, select clones with heightened antibody-dependent cellular cytotoxicity (ADCC) activity and assess their functionality in multiple murine tumor models.

An increased density of T regulatory (TREG) cells in tumors has been linked to poor survival outcomes15. In noncancer settings, TREG cells have been shown to differentiate into PD-1 expressing follicular regulatory T (T_{FR}) cells that restrain germinal center responses by suppressing B cells and T follicular helper (TFH) cells. T_{FR} cells moreover impede humoral immunity towards self-antigens and display heightened suppressive capacity when compared to TREG cells19,20. T_{FR} cells are being characterized by their joint expression of the surface molecules CXCR5 and GITR, or by their co-expression of the transcription factors FOXP3 and BCL-6. Critically, T_{FR} cells, their functional role in cancer, and their responsiveness to immunotherapy drugs have been completely disregarded so far. The inventors found that T_{FR} cells were prevalent in tumor tissues of several cancer types and exhibited superior suppressive capacity and in vivo persistence when compared to TREG cells, with whom they shared a clonal and developmental relationship. Crucially, it was found that T_{FR} cells, but not TREG cells, were enriched within TLS, showing that T_{FR} cells might also impair patient survival and impede immunotherapy treatment efficacy by regulating tertiary lymphoid structures (TLS), consistent with their well-described role in secondary lymphoid organs. Moreover, among tumor-infiltrating lymphocytes, T_{FR} cells expressed the highest levels of CTLA-4 and PD-1, making them susceptive to ICB targeting these molecules. Accordingly, in a genetic murine model permitting selective T_{FR} cell depletion, the inventors found that T_{FR} cells curtail anti-PD-1 treatment efficacy, providing ample rationale to study the role of T_{FR} cells in shaping anti-tumor immunity also with regard to anti-PD-1 therapy. Cancer vaccines have emerged as one of the leading strategies to augment anti-tumor immunity. They target tumor-associated antigens, neoantigens or viral antigens to induce tumor regression, eradicate small tumors and induce a long-lasting anti-tumor immune response. Various strategies to improve vaccine efficacy exist and are typically based on strategies to augment DC activity or target specific DC subsets in draining lymph nodes. Direct antigen delivery strategies include peptide vaccines, RNA vaccines and DNA vaccines, that rely on antigen cross-presentation by cDC1 cells. CD4+ T cells are critical facilitators of efficacious cancer vaccines as they ‘license’ cDC1 cells for priming of CD8+ T cell responses. This process depends upon direct cell-cell contacts between CD4+ T cells and cDC1s that drive DC activation. Conversely, TREG cells have been shown to induce cross-tolerization, a process that depends on TREG—cDC1 interactions and which reduces cDC1 activity to promote tolerance to a given antigen. Thus, the ratio of CD4+ T cells to TREG cells might be an important regulator of cDC1 activity and thus anti-tumor immunity. Accordingly, in T_{FR} cell-deficient mice, the inventors found a significantly increased abundance of cDC1 cells, thus, T_{FR} cells might regulate the priming of anti-tumor CD8+ T cell responses and impact cancer vaccine efficacy.

Immunotherapies have become crucial treatment options for a variety of cancer types and several novel targets like TIM-3, TIGIT, GITR, VISTA, LAG3 or ICOS are currently being explored to evaluate their antitumor capacity. Crucially however, most of these targets suffer from on-target, off-cell effects, as other immune cell types can express high levels of these molecules. Accordingly, the inventors have shown that intratumoral PD-1 expressing follicular regulatory T (T_{FR}) cells are critical determinants of anti-PD-1 treatment efficacy1, and that anti-PD-1 therapy can activate such suppressive cells, thus dampening treatment efficacy. In line with this, it has been demonstrated that the balance of PD-1 expressing CD8+ T cells and T regulatory (TREG) cells in the tumor microenvironment (TME) is a critical biomarker predicting anti-PD-1 treatment efficacy. Similarly, TREG cells have been shown to be activated by agonistic anti-4-1BB antibodies. These findings show that immunotherapy target expression levels on different immune cell types need to be carefully evaluated to determine which patients might benefit from a given treatment. Furthermore, while established immunotherapy drugs like anti-PD-1 or anti-CTLA-4 have shown remarkable success in some instances, only a fraction (˜20%) of patients respond to treatment. It is well appreciated that anti-CTLA-4/anti-PD-1 combination therapy results in significantly higher overall response rates compared to monotherapy with either agent, but that combination therapy also induces more frequent and severe irAEs, thus limiting its use. Hence, as off-cell effects and widespread immune related toxicity severely limit both treatment efficacy and combination therapy options, there is urgent need to develop novel immunotherapy targets that exhibit a more restricted expression profile. The inventors have identified IL1R2, a nonsignaling decoy receptor for the cytokine Interleukin-1b, to be highly expressed by T_{FR} cells in humans and mice. Unlike other TREG depletion strategies currently in clinical use (CTLA-4) or clinical development (CD25, CCR8), which might cause irAEs due to their broad or constitutive expression by TREG cells in multiple non-malignant tissues9, IL1R2 exhibits a more restricted expression profile. As such, anti-IL1R2 antibodies might deplete suppressive intratumoral T_{FR} cells more selectively than other approaches, thus combining high treatment efficacy with potentially lower toxicity.

Cancer vaccines have been shown to rely on cross-presentation of the immunizing tumor-associated antigen, viral antigen or tumor neoantigen via cDC1s. CD4+ T cells are critical facilitators of vaccine efficacy as they ‘license’ cDC1 cells for priming of CD8+ T cell responses. This process depends upon direct cell-cell contacts between CD4+ T cells and cDC1s that drive DC activation. Conversely, TREG cells induce cross-tolerization, a process that depends on TREG-cDC1 interactions and which reduces cDC1 activity to promote tolerance to a given antigen. Thus, the ratio of CD4+ T cells to TREG cells (and therefore T_{FR} cells) is likely to be an important regulator of cDC1 activity and thus antitumor immunity. Because cDC1s presenting tumor antigen epitopes can either interact with a CD4+ T cells or a T_{FR} cell under steady state conditions, depletion of T_{FR} cells will also improve co-stimulation of tumor-antigen specific CD4 T cells. Therefore, the inventors expect that depletion of TFR cells prior to vaccination will increase the likelihood and length of CD4+ T cell-cDC1 cell interactions, thus licensing cDC1 cells for effective CD8+ T cell priming.

Assessing impact of selective T_{FR} cell depletion on anti-tumor immunity and anti-PD-1 treatment efficacy. Strategy and Methods. To determine effects of selective depletion of TFR cells, the inventors can use genetic and therapeutic (anti-CTLA-4 or anti-IL1R2) approaches for (a) combined depletion of TREG and T_{FR} cells and (b) selective depletion of T_{FR} cells.
(i) Genetic approach: Foxp3DTR mice permit the inducible (transient or long-term) depletion of all FOXP3-expressing (TREG and T_{FR} cells). Heterozygous and homozygous Foxp3YFP-cre×Bcl6fl/fl mice partially or completely deplete FOXP3+ cells that express Bcl-6 (T_{FR} cells).
(ii) Therapeutic approach: The inventors have shown that anti-CTLA-4 therapy nonspecifically depletes both TREG and T_{FR} cells. Conversely, the inventors predict that anti-IL1R2 antibodies will selectively deplete T_{FR} cells due to its exclusive expression in T_{FR} cells, which the inventors can test. It is possible to test the effects of (a) combined depletion of TREG and T_{FR} cells and (b) selective depletion of T_{FR} cells in tumor models (B16F10-OVA and MC38-OVA) with and without anti-PD-1 therapy.

### Assessing Impact of Selective T_{FR} Cell Depletion on Efficacy of Cancer Vaccines.

Strategy and Methods. Tumor model to test cancer vaccines. It is possible to use TC-1 tumor cells, which express HPV-16 E7 associated antigens and hence induce immune responses against the HPV E7 protein. Others have shown that a DNA vaccine encoding a long peptide sequence from within the E7 protein induces HPV E7-specific (RAHYNIVTF tetramer) CD8+ T cell responses that rely on CD4+ T cell help10-14. The inventors have validated this tetramer in preliminary studies in tdLNs utilizing a low-dose vaccine. Thus, this model is suitable for testing effects of T_{FR} cell depletion on cDC1-CD4+ T cell interactions, CD4+ T cell help, and priming and function of CD8+ T cells. To evaluate the impact of TREG and T_{FR} cells on cancer vaccine efficacy, the inventors can inoculate mice with TC1 tumor cells in the above mentioned TREG and/or T_{FR} depleting experimental conditions. To assess whether TREG and T_{FR} cells impede priming, the inventors can deplete the cells of interest after tumor inoculation but prior to intramuscular vaccination with the DNA vaccine encoding for long peptide sequences of the HPV16 E7 protein.

Assessments and analyses. The assessments are as outlined above. Briefly, the inventors can assess whether T_{FR} cell-deficiency (genetic or pharmaceutical approach) alters the priming of CD8+ T cells (i) (FIG. 3, early assessment). The inventors can assess the impact on DC subsets (ii) (frequency, priming and activity, FIG. 3 early and late assessment) in primary tumor tissue and tdLNs, measure tumor growth (iii), quantify and qualify tumor-infiltrating T cells (iv) and perform transcriptomic analyses (v). (vi) cDC1 licensing. To further demonstrate that the alterations in anti-tumor immunity are dependent on cDC1-CD4+ T cell interactions, the inventors can additionally deplete CD4+ T cells with anti-CD4 antibodies. By way of explanation, but in no way a limitation of the present invention, the inventors hypothesize that MHC-II peptides and MHC-I peptides don't have to stem from the same antigen (as long as the source antigens are processed by the same DC), as any functional interaction between CD4+ T cells and cDC1s could license the cDC1 cells for priming of CD8+ T cells. The inventors can adoptively transfer OT-II T cells (CD4+ T cells with a TCR specific for Ovalbumin) and OVA-peptide pulsed cDC1 cells in the TC1 model prior to vaccination.

As a lack of T_{FR} cells led to an increased frequency of migratory cDC1 cells in tumor-draining lymph nodes, TFR cell depletion is expected to improve priming (i.e., clonal expansion or epitope spreading) of anti-tumor CD8+ T cell responses in a cancer vaccine setting. As the data show the clinical benefit of sequential immunotherapy (depleting intratumoral T_{FR} cells prior to initiation of anti-PD1 therapy), a T_{FR} cell depletion prior to vaccination will have similarly beneficial effects as it would improve the likelihood of functional cDC1-CD4+ T cell interactions.

### Example 3. Intermittent PI3Kδ Inhibition Sustains Anti-Tumor Immunity and Curbs irAEs

As described above, phosphoinositide 3-kinase δ (PI3KS) plays a key role in lymphocytes and inhibitors targeting this PI3K have been approved for B cell malignancies¹⁻³. While studies in murine solid tumor models have demonstrated that PI3Kδ inhibitors (PI3Kδi) can induce anti-tumor immunity⁴, its impact on solid tumors in humans remains unclear. Here, the inventors assessed the effects of the PI3Kδi AMG319 in patients with head and neck cancer in a neoadjuvant, double-blind, placebo-controlled randomized phase-II trial (EudraCT #2014-004388-20). PI3Kδ inhibition decreased tumor-infiltrating T_{REG} cells and enhanced the cytotoxic potential of tumor-infiltrating T cells. At the tested AMG319 doses, immune-related adverse events (irAEs) required treatment discontinuation in 12/21 of AMG319-treated patients, suggestive of systemic effects on T_{REG} cells. Accordingly, in murine models, PI3Kδi decreased T_{REG} cells systemically and caused colitis. Single-cell RNA-seq analysis revealed a PI3Kδi-driven loss of tissue-resident colonic ST2 T_{REG} cells, accompanied by expansion of pathogenic T_{H}17 and T_{C}17 cells, likely contributing to toxicity and pointing towards a specific mode of action for the emergence of irAEs. A modified treatment regimen with intermittent dosing of PI3Kδi in murine models led to a significant decrease in tumor growth without inducing pathogenic T cells in colonic tissue, indicating that alternative dosing regimens might limit toxicity.

PI3K inhibitors were initially considered to mainly target cancer cell-intrinsic PI3K activity, which was the underlying rationale to test inhibitors against the leukocyte-enriched PI3Kδ in B cell malignancies. However, subsequent studies have shown that PI3Kδ inhibition also has clear immunomodulatory activities, largely T cell-mediated, which were under-appreciated at the time of the early trials in B cell malignancies, causing irAEs that have hampered clinical progress and utility. Several lines of evidence suggest that PI3Kδi preferentially inhibit T_{REG} cells over other T cell subsets⁴⁻⁸ but to date, no trials have been performed to explicitly explore this concept in humans. The current study provides the first in-depth investigation of the impact of PI3Kδ inhibition on immune cells in patients with solid tumors and also explores the mechanism leading to irAEs.

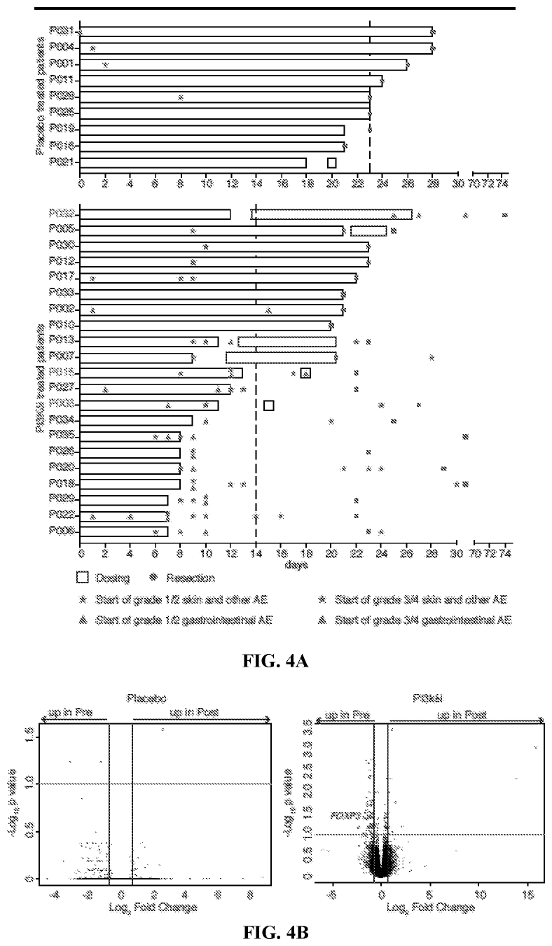

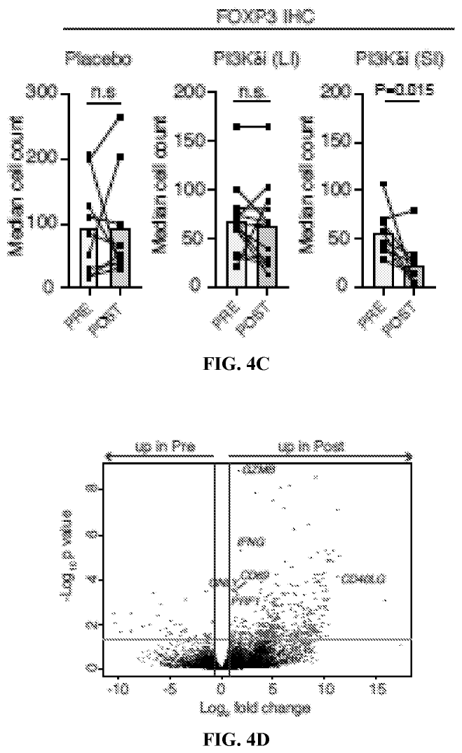

PI3Kδ inhibition causes irAEs. To evaluate the potential for PI3Kδ inhibitors as immunotherapeutic agents in human solid cancers, the inventors administered the PI3Kδi AMG319 to treatment-naive patients with resectable head and neck squamous cell carcinoma (HNSCC) in a neoadjuvant, double-blind, placebo-controlled randomized phase II trial. The inventors measured target inhibition (pAKT levels in B cells and drug levels to verify drug administration. Thirty-three patients were randomized in a 2:1 ratio (AMG319:Placebo) to the trial and 30 patients received at least one dose of AMG319 or placebo. Fifteen patients received 400 mg daily of AMG319 (range of 7-24 days per patient). Unexpectedly, at the 400 mg dose, 9/15 patients experienced irAEs that lead to withdrawal of treatment. After a formal safety review, 6 additional patients were recruited and treated at a reduced dose of 300 mg per day. Again, 3/6 patients had irAEs that led to discontinuation of treatment. One patient experienced grade 4 colitis after completion of 24 daily doses of AMG319 and eventually required colectomy (FIG. 4A). The most prevalent irAEs were skin rashes (29%; 25% observed in the treatment group and 4% in placebo group), diarrhea (29%; 28% in observed treatment group and 1% in placebo group) and transaminitis (14% all in the treatment group), consistent with a treatment-mediated loss of T_{REG} cells or T_{REG} cell functionality in multiple tissues causing immunopathology. The onset of irAEs was surprisingly rapid (median time to onset 9 days) and led to treatment discontinuation in 12/21 AMG319-treated patients. Clinically, and most likely reflecting the brief treatment period, the inventors did not observe any significant differences in the measured tumor volumes between the study arms in the 23 patients in whom this was evaluable. Two patients with partial responses (PR) and one with complete pathological response occurred in AMG319-treated patients, all of whom also exhibited grade 3/4 irAEs.

PI3Kδi alter the tumor microenvironment. Whole tumor RNA-seq analysis of pre- and post-treatment tumor samples revealed substantial differences in the AMG319 treatment group (93 differentially-expressed genes (DEGs)), but not for the placebo group (3 DEGs)(FIG. 4B). As PI3KS-inhibition led to a significant reduction in FOXP3 transcript levels in the tumor samples (FIG. 4B), the inventors assessed T_{REG} cell levels in tumor tissue via immunohistochemistry, hypothesizing that the duration between ceasing of treatment and tumor resection might be critical factor influencing T_{REG} cell abundance due to the relatively short half-life of the compound. Indeed, the inventors found significantly reduced intratumoral T_{REG} cells only in patients in which their abundance could be assessed directly after treatment (PI3Kδi short interval) (FIG. 4C), showing that T_{REG} cell levels normalize quickly once treatment has been stopped.

Bulk-RNA-seq analysis of sorted tumor-infiltrating CD8⁺ T cells revealed higher expression of IFNG, GZMB and PRF1 in post-treatment samples, indicating enhanced cytotoxic potential of tumor-infiltrating CD8⁺ T cells following PI3Kδi treatment (FIG. 4D). The inventors corroborated these results with single-cell RNA-seq analysis, which demonstrated that CD4⁺ and CD8⁺ T cell clusters showed a treatment-associated increase in expression of cytotoxicity genes (e.g., GZMB and PRF1). The inventors also found a modest clonal expansion of CD4⁺ and CD8⁺ T cells post-treatment. As low cell numbers for CD4⁺FOXP3⁺ T cells (n=0-27 cells per patient) precluded a more detailed analysis in this cohort, the inventors next assessed circulating T_{REG} cells. PI3KS-inhibition led to a significant increase in activated circulating T_{REG} cells, while the proportion and activation status of T_{REG} cells in the placebo group remained stable. This implies that PI3Kδ inhibition either influences proliferation, or that it displaces activated T_{REG} cells from tissues, presumably by altering the expression of tissue homing factors like KLF2 and S1PR1, direct targets of FOXO1 in line with previous studies⁵⁻⁷, likely contributing to toxicity. Together, these data indicate that PI3Kδ inhibition causes profound changes in the tumor microenvironment (TME), characterized by enhanced CD4⁺ and CD8⁺ T cell activation, oligoclonal T cell expansion and increased cytolytic activity, consistent with a decrease in intratumoral T_{REG} cells, enabling T cell activation, but also leading to a rapid onset of dose-limiting toxicity.

Systemic effects of PI3Kδi on T_{REG} cells. To understand the mechanistic basis of PI3Kδi-induced toxicity and anti-tumor immune responses, the inventors next tested the impact of a PI3Kδi in a mouse solid tumor model. C57BL/6 wild-type mice were inoculated with B16F10-OVA melanoma cells and treated with the previously described PI3Kδi PI-3065⁷. Consistent with previous studies^{4,5}, the inventors found a significant decrease in tumor volume and a significant increase in intratumoral CD8⁺ T cells that expressed high levels of PD-1 and exhibited increased proliferative and cytotoxic capacity. TOX, a transcription factor recently identified as critical for adaptation and survival of CD8⁺ T cells in the tumor microenvironment (TME)⁹, was also increased post-PI3Kδi. Notably and contrary to previous reports^{10,11}, the inventors found that the expression of both granzyme B and Ki-67 was almost exclusively limited to TOX*CD8⁺ T cells, demonstrating that these cells, despite showing high expression of PD-1 and TOX, are not functionally exhausted in this tumor model.

Given that PI3K inhibitors were initially considered to mainly target cancer cell-intrinsic PI3K activity, the inventors utilized RAG1^{-/-} and CD8^{-/-} mice to verify that the observed anti-tumor effects were dependent on immune cells, and more specifically on CD8⁺ T cells. As PI3Kδ inhibition caused substantial irAEs in non-malignant organs (FIG. 4B) and given that T_{REG} cells have been shown to be susceptible to this form of treatment, the inventors next assessed whether PI3Kδi act locally within the tumor tissue or systemically. Importantly, in PI3Kδi-treated mice, but not placebo-treated control mice, the inventors found a significant decrease in T_{REG} cells in tumor, spleen and colon, indicative of systemic effects of PI3Kδi on T_{REG} maintenance or survival.

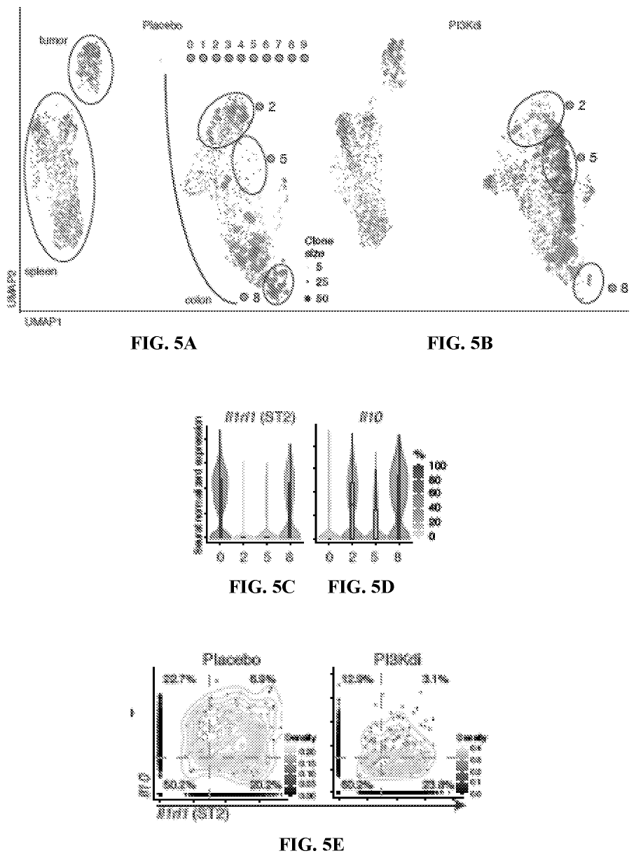

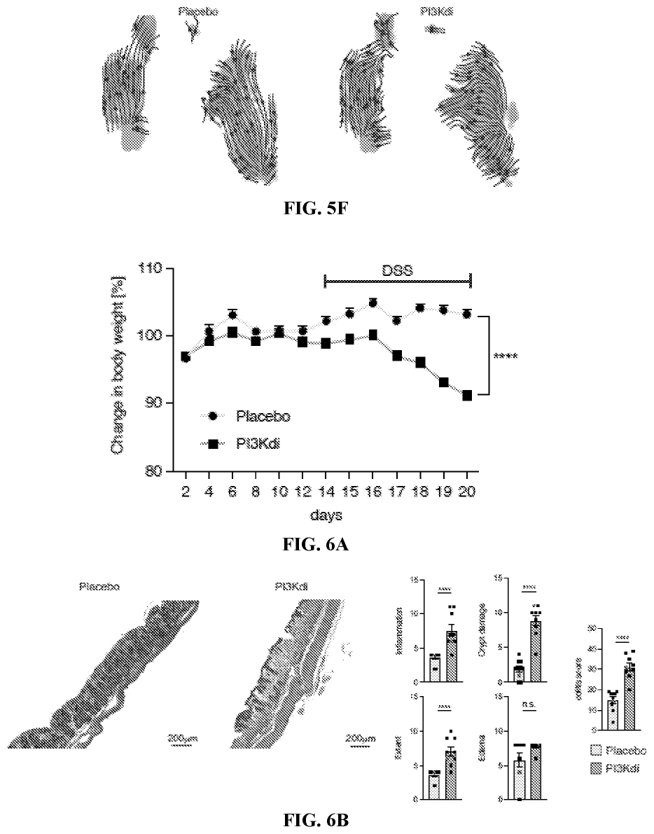

PI3Kδi affect specific T_{REG} cell subsets. Since gastrointestinal toxicity is one of the major irAEs in patients receiving PI3Kδi^{4,6,12} (FIG. 4B), the inventors hypothesized that T_{REG} cells present in colonic tissue may be especially sensitive to PI3Kδi. To test this hypothesis in an unbiased manner, the inventors performed single-cell RNA-sequencing of T_{REG} cells isolated from tumor, spleen (lymphoid organ) and colonic tissue of PI3Kδi- and placebo-treated B16F10-OVA tumor-bearing mice. UMAP analysis identified 10 T_{REG} cell clusters, showing substantial T_{REG} cell heterogeneity and tissue dependent adaptations (FIGS. 5A, 5B), supporting the notion that several distinct T_{REG} subtypes exist in different locations, in agreement with previous studies^{13,14}. Colonic T_{REG} cells exhibited the most pronounced differences between PI3Kδi and placebo treatment, with 869 differentially expressed genes, while splenic and tumor T_{REG} cells exhibited fewer differences. Two of the colonic T_{REG} subsets (clusters 2 and 8) were depleted in PI3Kδi-treated mice (FIGS. 5, 5B). Cluster 2 colonic T_{REG} cells were enriched for the expression of Ctla4 and genes encoding chemokine receptors (Ccr1, Ccr2, Ccr4), critical for their suppressive^{15,16} and migratory capacity, respectively. Cluster 8 colonic T_{REG} cells, which showed substantial clonal expansion in control-treated mice but were depleted in PI3Kδi-treated mice, resembled the recently described tissue-resident ST2 T_{REG} cells¹⁸⁻²⁰, which are critical for the protection against chronic inflammation and facilitation of tissue repair (FIGS. 5A, 5B). Accordingly, the inventors found enrichment in the expression of the ST2 T_{REG} signature genes Illrl1 (IL-33R/ST2), Gata3 and Id2, as well as of several genes associated with highly suppressive effector T_{REG} cells (Klrg1, Cd44, Cd69, Pdcd1, Areg, Nr4a1, Ill0 and Tglb1). The inventors verified ST2 expression on T_{REG} cells at the protein level and found that PI3Kδ inhibition led to a substantially increased ratio of CD8⁺ T cells to ST2 T_{REG} cells. While colonic T_{REG} cells in cluster 0 and cluster 8 shared this ST2 signature (FIG. 5C), only cells in cluster 8 showed high transcript expression of the immunosuppressive cytokine IL-10 (FIG. 5D). These T_{REG} cell clusters (2 and 8) with highly suppressive properties were depleted in PI3Kδi-treated mice, while the clonally-expanded cluster 5 T_{REG} cells were enriched in PI3Kδi-treated mice showed a lack of transcripts associated with suppression (FIG. 5E) and instead higher expression of several interferon-related response genes (Stat1, Stat3, Ifrd1)^{21,22}, suggestive of a pro-inflammatory environment. Accordingly, ST2*Ill0⁺ T_{REG} cells were substantially reduced in PI3Kδi-treated mice (FIG. 5E). Interestingly, RNA velocity analysis, a tool to assess the developmental stage of cells in scRNA-seq data^{23,24}, infers a developmental trajectory over several progenitor states (cluster 2,4 and then 0) culminating in clonally-expanded ST2 T_{REG} cells (cluster 8) in placebo-treated mice (FIG. 5F). These data indicate that PI3Kδ inhibition prevents the cellular differentiation into ST2 T_{REG} cells, and instead diverts development to cluster 5 T_{REG} cells that lack expression of transcripts associated with suppressive capacity, pointing to a possible mechanism for the onset of inflammation and colitis. The inventors also observed a significant increase of CD8⁺ T cells in colonic, but not splenic tissue. Colonic CD8⁺ T cells expressed higher levels of PD-1 and ICOS upon PI3Kδ inhibition, showing treatment-related changes to cell activation. These findings show a heightened sensitivity of certain colonic T_{REG} subsets to PI3Kδi, potentially related to the high incidence of colitis observed in patients treated with PI3Kδ inhibitors.

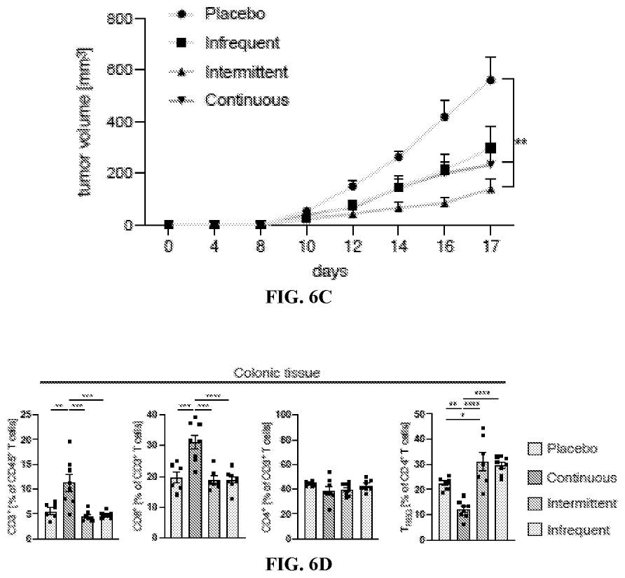

PI3Kδ inhibition exacerbates colitis. To explore the connection between PI3Kδ inhibition and gastrointestinal toxicity in more detail, the inventors utilized a Dextran Sulfate Sodium (DSS)-induced acute colitis model. Crucially, when compared to placebo-treated mice, the inventors found that PI3Kδ inhibition led to an accelerated and exacerbated disease phenotype, with a swift reduction in body weight and a higher overall colitis score characterized by significantly higher inflammation, crypt damage and area of infiltration (extent) (FIG. 6A, 6B), indicative of treatment-mediated alterations in tissue homeostasis driving immunopathology. To circumvent the emergence of these irAEs, the inventors hypothesized that a transient depletion of T_{REG} cells might suffice to restrict the immunosuppressive milieu in the tumor and thus drive anti-tumor immunity without causing substantial toxicity in non-malignant organs. The inventors tested this hypothesis by utilizing distinct treatment regimens, where mice would either be kept on PI3Kδi for the duration of the experiment (continuous dosing), be kept on PI3Kδi for 4 days followed by 3 days off drug (intermittent dosing) or be kept on PI3Kδi for 2 days followed by 5 days off drug (infrequent dosing) for a total of 2 treatment cycles (FIG. 6C). Strikingly, the inventors found that all treatment conditions led to a decrease in tumor growth, albeit not significantly for the infrequent dosing condition, suggesting that transient interruptions of the immunosuppressive TME drive anti-tumor immunity. Most importantly, only continuous dosing led to increased CD8⁺ T cell infiltration and decreased T_{REG} cell levels in colonic tissue (FIG. 6D), indicating that intermittent dosing regimens might decrease irAEs in human also.

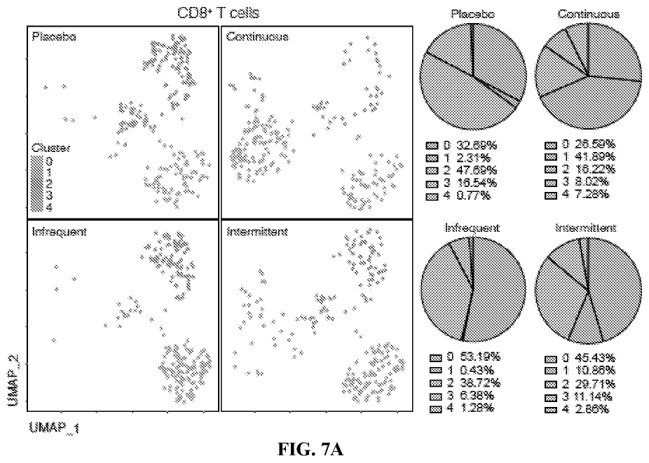

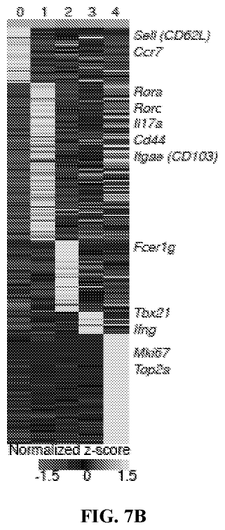

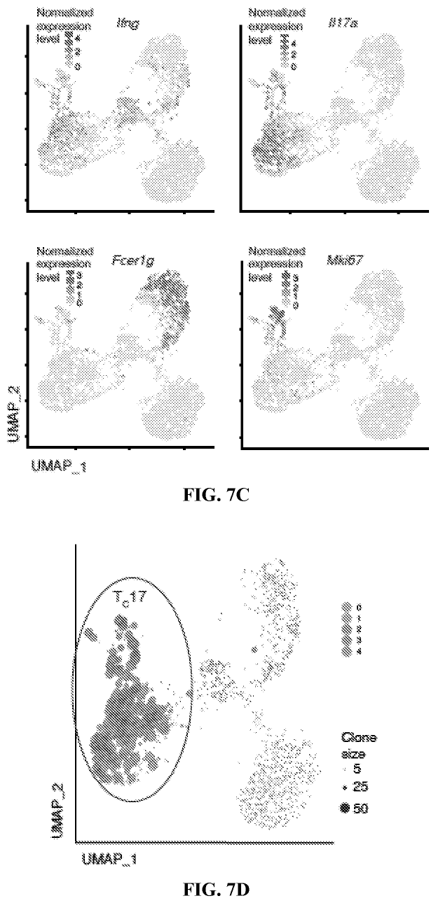

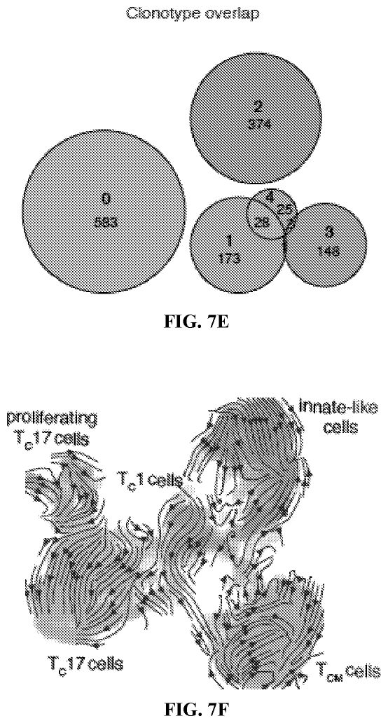

Intermittent-dosing curbs toxicity. To discern whether specific T cell subsets drive immunopathology upon PI3Kδ inhibition, the inventors performed scRNA sequencing of colonic CD8⁺ and CD4⁺ T cells in the different treatment regimens. Unbiased clustering depicted by UMAP revealed 5 distinct CD8⁺, and 6 distinct CD4⁺ T cell clusters (FIG. 7A). In both instances, the inventors identified a central memory T_{CM} subset (cluster 0, red) expressing high levels of Ccr7 and Cd62L, a T_{C}1 and T_{H}1 subset expressing high levels of interferon-γ transcripts (cluster 3, T_{C}1 and cluster 1, T_{H}1), a T_{C}17 and T_{H}17 subset enriched for 11-17 transcripts (cluster 1, T_{C}17 and cluster 2, T_{H}17), and a proliferative subset that exhibited features of T_{C}17 or T_{H}17 cells, respectively (FIG. 7B, 7B). Strikingly, the inventors found a dosing-dependent enrichment of the T_{C}17 and T_{H}17 subsets and pertaining proliferating clusters, making up ˜50% of all cells in the continuous dosing regimen, while they were nearly completely absent in the other treatment conditions (FIG. 7A). Importantly, IL-17 producing cells have been shown to cause colitis²⁵⁻²⁷. Cells in these Il-17⁺ clusters were moreover heavily clonally expanded and exhibited substantial cellular and clonotypic overlap in both CD8⁺ and CD4⁺ T cells (FIG. 7D, 7E), likely contributing to their rapid expansion. Conversely, the inventors found a dosing-dependent decrease of innate-like CD8⁺ T cells, which have been implicated in controlling inflammation and the onset of colitis^{28,29} (FIG. 7A-7C). Lastly, RNA velocity analyses shows that the pathogenic T_{C}17 and T_{H}17 subsets derive from IFN-γ-expressing progenitor cells (FIG. 7C, 7F). Accordingly, T_{C}17 and T_{H}17 maintained high transcript expression of IFN-γ (FIG. 7C).

Given that IL-10+ST2 T_{REG} cells have been implicated in controlling IL-17 responses that would otherwise cause colitis³⁰, these data provide a novel and rational explanation for the ripple effects ensuing after PI3Kδ inhibition that eventually cause irAEs. Specifically, these data show that Ill0-expressing ST2 T_{REG} cells are highly susceptible to PI3Kδ inhibition, leading to a decrease in their abundance and thus to a disruption of gut homeostasis by causing a rapid expansion of pathogenic T_{H}17 and T_{C}17 cells that, together with a decrease in innate-like CD8⁺ T cells, cause colitis. Intermittent PI3Kδi dosing moreover provides the means to uncouple the anti-tumor effects from irAEs, providing ample rationale to test this concept in a follow-up clinical trial.

It was found that in human and mouse tumor tissue, PI3Kδ inhibition leads to substantial changes in the cell composition of the TME by reducing T_{REG} cells and activating intratumoral CD4⁺ and CD8⁺ T cells, which clonally expand and display heightened cytotoxic and cytolytic features. Notably, in mouse models, the inventors found substantial changes in the transcriptional features and composition of colonic T_{REG} cell subsets, which indicate that PI3Kδ inhibition impacts T_{REG} functionality, survival and tissue retention, thus altering T_{REG} cell frequencies or T_{REG} subtype compositions in both tumor and non-malignant tissues. These treatment-mediated changes, specifically the depletion of Ill0-expressing ST2 T_{REG} cells is associated with colitis and expansion of pathogenic T_{C}17 and T_{H}17 T cell subsets in colonic tissue. Notably, these findings are more broadly applicable, as tissue-resident ST2 T_{REG} cells have been described in many non-malignant organs frequently affected by irAEs (i.e., skin) or might be affected by other T_{REG} cell targeting immunotherapies (i.e., anti-CTLA-4). The inventors showed in mouse models that intermittent dosing with PI3Kδi is a rational treatment strategy that combines sustained anti-tumor immunity with reduced toxicity.

These data show that the immunomodulatory effects of PI3Kδi need to be evaluated judiciously in treatment-naïve patients, unaffected by multiple lines of treatment and the immunosuppressive effect of hematological malignancies such as CLL. It is clear that in the neoadjuvant setting in patients with HNSCC, at the evaluated doses and with daily scheduling, PI3Kδ inhibition exhibits an unfavorable safety profile, limiting its feasibility and clinical benefit by causing frequent and severe grade 3/4 irAEs, most likely driven by modulation of T_{REG} cell behavior in non-malignant tissues. Based on these findings, decreased dosages or an altered PI3Kδi treatment regimen will be required in solid tumors, especially in immune-competent patients, in order to be able to exploit the clear anti-tumor immune response induced by PI3Kδi while limiting the adverse effects associated with reduced T_{REG} function in healthy tissues. Finally, these data show that the unique cellular composition of effector versus regulatory cells in the TME of each patient might be an important determinant of the efficacy of PI3Kδ inhibition. As such, PI3Kδi might be especially useful in patients with high levels of intratumoral T_{REG} cells and an unfavorable ratio of T_{REG} versus CD8⁺ TILs in pre-treatment samples. Hence, this study shows PI3Kδ inhibitors (PI3Kδi) as immunomodulatory agents in solid tumors.

Double blind, randomized clinical trial and sample collection. In order to explore the immunomodulatory effects of PI3Kδ inhibition in humans, the inventors conducted a multicenter, placebo-controlled phase II neoadjuvant trial with the PI3Kδi AMG319 in resectable HNSCC, www.clinicaltrialsregister.eu/ctr-search/trial/2014-004388-20/results). All patients have provided written informed consent for participation in the clinical trial. The inventors studied Human Papilloma Virus (HPV)-negative HNSCC, as this cancer type is more prevalent, and because patients with this cancer type have poorer outcomes when compared to HPV-positive HNSCC, likely due to overall lower tumor-infiltrating lymphocyte (TIL) infiltration³¹⁻³³. The clinical trial was sponsored by Cancer Research UK Center for Drug Development (CRUKD/15/004) and approved by the Southampton and South West Hampshire Research Ethics Board; the trial EudraCT number is 2014-004388-20. Detailed information about the trial design, randomization procedure, protocol amendments, recruitment data, patient characteristics and adverse events are deposited at www.clinicaltrialsregister.eu/ctr-search/trial/2014-004388-20/results #morelnformationSection and found in the CONSORT checklist. Patients were recruited after initial diagnosis and before definitive surgical treatment; drug treatment or placebo was given for up to 24 or 28 days respectively, prior to resection of tumor. In a previous phase I dose-escalation study of heavily-pretreated patients with either CLL or non-Hodgkin lymphoma, AMG319 doses of up to 400 mg were explored without reaching a maximally-tolerated dose, and exhibited PK dynamics with a mean half-life of 3.8-6.6h in plasma³⁴. In that phase 1 study, daily dosing with 400 mg AMG319 led to near-complete target inhibition (BCR-induced pAKT in ex vivo IgD-stimulated CLL samples) and >50% nodal regression³⁴, while immune-related adverse events (irAE) at grade 3 or above according to the common toxicity criteria (CTC) occurred after days 40 and 60. The inventors reasoned that high-grade irAEs were unlikely to occur during the shorter treatment duration in the neoadjuvant setting, and therefore selected 400 mg/day as the starting dose. The intended time from initiating treatment with AMG319 or placebo to surgical resection of tumor was up to 4 weeks, with weekly blood draws. The full evaluation of radiological measurements has previously been reported at https://www.clinicaltrialsregister.eu/ctr-search/trial/2014-004388-20/results to the EU Clinical Trials Register in compliance with regulatory requirements. Primary endpoints were safety and assessment of CD8⁺ immune infiltrates, secondary endpoints tumor responses and AMG319 pharmacokinetic evaluation (https://www.clinicaltrialsregister.eu/ctr-search/trial/2014-004388-20/results#endPointsSection). The sample size was calculated as follows: in a pilot cohort, the CD8 count in the biopsy taken at diagnosis, and in the resected tissue sample was quantified. The mean value at diagnosis was 25 cells/high power filed (hpf), and this remained almost the same in the resected sample (26 cells/hpf). With an observed standard deviation of five cells it was posited that a doubling to 50 cells/hpf following treatment with AMG 319 would be observed, hence a difference between the two treatment groups of 25. To detect a standardized difference of 0.5 with 80% power and one-sided test of statistical significance of 20%, 36 patients were randomized to AMG319 and 18 to placebo (54 in total). Randomization was at the level of the individual patient, using block randomization with randomly varying block sizes. During the course of the clinical trial the randomization list was held by the unblinded Trial Statistician and within the IWRS. Patients and care providers were blinded to the treatment allocation, and all immunological evaluations were completed by a pathologist and researchers who were blinded to the patient allocation to treatment arms. Patients were recruited from October 2015 to May 2018 at in the UK (University Hospital Southampton NHS Foundation Trust, Poole Hospitals NHS Foundation Trust, Liverpool University Hospitals NHS Foundation Trust and Queen Elizabeth University Hospital Glasgow, two additional centers did not recruit patients); written informed consent was obtained from all subjects. Patients were eligible if they were >18 years, with histologically proven HNSCC for whom surgery was the primary treatment option, with laboratory results within specified ranges. Patients had to be clinically eligible for tumor resection; patients who had undergone prior radio/immuno/chemotherapy or other anti-cancer therapy for their current HNSCC, were excluded. Clinical data were obtained for age, gender, tumor size (T stage), and nodal status (N stage) (summarized in Source Data_Patient characteristics). Adverse event reporting was according to the National Cancer Institute CTCAE Version 4.02. Performance status and overall survival was collected to death or censored at last clinical review; clinical data were anonymized once the data had been collated and verified by the sponsor. Drug dosing was at 400 mg of the oral PI3Kδ inhibitor AMG319 (15 patients) and, after an independent safety review, dosing at 300 mg in 6 patients; all patients who had at least 4 doses of the drug were included in the final analyses. Radiological evaluation of change in tumor volume (ED FIG. 1e) was undertaken by comparing baseline bi-dimensional measurements of tumor at baseline and before surgery. For Response assessment RECIST 1.1 was used. The full data on radiological measurements is available at https://www.clinicaltrialsregister.eu/ctr-search/trial/2014-004388-20/results in the EU Clinical Trials Register in compliance with regulatory requirements. The study was discontinued after 30 (of the target sample size of 54) patients had been dosed with AMG319 or placebo, thus limiting the clinical information on outcomes that can be gained from this trial. All patients had tissue collected as a dedicated research biopsy after consent and prior to randomization, with an additional sample collected during surgical resection. Tumor tissue was obtained fresh on the day of biopsy/surgery and a sample was immediately snap frozen. A proportion of the tumor tissue was cryopreserved in freezing medium (90% FBS and 10% DMSO) for subsequent analyses or, alternatively, directly disaggregated using a combination of enzymatic and mechanical dissociation for immediate analysis by FACS or cryopreservation as a single cell suspension, as previously described³⁵. Blood samples were collected during the course of the study from which plasma and PBMCs were collected. PBMCs were isolated by centrifugation over Lymphoprep™ (Axis-Shield PoC AS).

Histology and Immunohistochemistry. Double immunostaining for CD8 and FOXP3 was performed on a Leica Bond RX platform, with antigen retrieval performed for 20 min at 97° C. Bond ER2 antigen retrieval solution. Primary antibodies were incubated for 30 mins at room temperature (FoxP3-Abcam: Clone 236A/E7 1:100 dilution; CD8-DAKO: Clone C8/144B 1:50 dilution) and detected using the Leica Refine Polymer brown and red detection systems. Analysis was performed by two independent and blinded head & neck pathologists counting intratumoral CD8+ and FoxP3+TIL in multiple random high-power fields (HPF) at magnification ×200. Where possible, 10 HPF were counted.

PK of AMG319. 50 μL of thawed plasma samples were mixed with 300 μL of Extraction Solution (100 ng/mL [2H3, N15]-AMG319 in methanol), centrifuged at 10,000 g/RCF for 5 minutes to precipitate the plasma proteins. The supernatant was transferred to a UPLC vial and placed on the autosampler (maintained at 8° C.) for analysis. A freshly prepared calibration curve in the range 1-1000 ng/mL and frozen QC samples at 10, 100, 500 and 1000 ng/mL (K2 EDTA human plasma spiked with AMG319) were analyzed alongside each batch of patient samples. 5 μL of supernatant was injected into the UPLC-MS/MS system, configured with a Waters Acquity UPLC and Waters Quattro Premier XE mass spectrometer. Analytes were separated on an Acquity UPLC BEH C18 1.7 gm (2.1 mm×100 mm) column with a mobile phase flow rate of 0.3 mL/min. Mobile phase was composed of water, acetonitrile and formic acid. Analytes were detected using the multiple reaction monitoring (MRM) mode of the MS/MS system, operating in positive ion electrospray mode. MRMs were set up at m/z 386.4>251.3, 386.4>236.6, 251.3>251.3 and 251.3>236.3 for AMG319 and at m/z 390.5>254.4 for [2H3, N15]-AMG319. MassLynx software (version 4.1, Waters Ltd.) was used to control the instrumentation and for analysis of the peaks of interest and processing of spectral data.

pAKT measurement. Whole blood samples (10 ml) were collected in sodium heparin tubes pre-dose and 4 hours post dose on days 1 and 15 for the first 11 patients (day 8 and 15 for the remaining 19 patients). Blood was stimulated with double-diluted anti-IgD (25-0.008 gg/ml) in deep well plates for 5 min. Blood was then lysed and fixed with BD PhosFlow Lyse/Fix buffer. Cell pellets were washed and then stored at −80° C. until all samples from the same patient were ready for further analysis. Upon thawing, cells pellets were incubated with anti-human CD3-FITC and CD14-FITC, washed PBS+1% FBS, permeabilised with 80% MeOH and washed again before intracellular staining with CD20-PE Cy7 and pAKT (S473). Stained cell pellets were washed again before staining with a secondary antibody (anti-rabbit Alexa 647). Events were subsequently acquired on a Canto II flow cytometer (BD), and analyzed using FACS Diva. MFI of pAKT in B cells was plotted against the anti-IgD concentration, which was used to activate the B cells. The AUC was calculated and a drop of 50% in AUC between pre- and post-dose was validated to be the result of drug inhibition.

Mice. C57BL/6J (JAX stock #000664), OT-I (JAX stock #003831), Rag1^{-/-} (JAX stock #002216) and CD8/(JAX stock #002665) mice were obtained from Jackson labs. Foxp3^{RFP} (JAX stock #008374) were a kind gift from K. Ley (LJI). Age (6-12 weeks) and sex-matched mice were used for all experiments. The housing temperature is controlled, ranging from 69-75 F, humidity is monitored but not controlled and ranges from 30-70%. The light/dark cycles are from 6 am-6 pm, respectively. All animal work was approved by the relevant La Jolla institute for Immunology Institutional Animal Care and Use Committee.

Tumor experiments. Mice were inoculated with 1-1.5×10⁵ B16F10-OVA cells subcutaneously into the right flank. Mice were put on either a control diet or a diet containing a PI3Kδ inhibitor PI-3065 on day 1 or day 5 after tumor inoculation. Diets were prepared using powdered 2018 global rodent diet (Envigo) mixed with or without PI-3065 at 0.5 g/kg, which corresponds to a daily dose of 75 mg/kg as used previously. To pellet the food, 50% v/w water was added to the diet and dough thoroughly mixed, compressed, molded and dried before use. Tumor size was monitored every other day, and tumor harvested at indicated time points for analysis of tumor-infiltrating lymphocytes. Tumor size limit of 15 mm in diameter was not exceeded and volume was calculated as ½×D×d², where D is the major axis and d is the minor axis, as described previously³⁶.

Bulk transcriptome analyses. Cryosections (10 gm) were cut from snap frozen tumor and RNA was extracted using the Maxwell® RSC instrument and Maxwell® RSC SimplyRNA Tissue kit (Promega, Southampton, UK), according to the manufacturer's instructions. RNA was quantified using the Qubit fluorometer (ThermoFisher Scientific) and quality was assessed using the Agilent 2100 Bioanalyzer generating an RNA integrity number (RIN; Agilent Technologies UK Ltd.). RNA sequencing was performed by Edinburgh Genomics (Edinburgh, UK.); mRNA libraries were prepared using the TruSeq Stranded Total RNA Library Prep Kit (Illumina) and paired-end sequenced (100 bp) on the NovaSeq 6000 platform (Illumina) to yield an average read depth of 40×10⁶ reads. Reads were mapped to hg19 reference genome using STAR github.com/ndu-UCSD/LJI_RNA_SEQ_PIPELINE_V2. A total of 22 paired (14 from treatment and 8 from placebo group) samples with at least 70 percent of mapping reads were selected. Differential expression analysis between the pre and post-treatment, as well as between pre and post placebo, was performed using DESeq2 (v1.24.0). The threshold for differentially expressed genes was determined with fold change of >log₂ 0.75 and an adjusted P-value <0.1. Between treatment pre and post, 93 genes were identified as significant, whereas 3 genes were significant between placebo pre and post. Cells were dispersed from fresh tumor tissue and used immediately for flow cytometric analysis and cell sorting. CD8⁺ T cells were bulk sorted into ice-cold TRIzol LS reagent³⁵ (Thermo Fisher Scientific) on a BD FACS Fusion (BD Bioscience). Reads from sorted CD8 RNA were mapped to hg19 reference genome using STAR with the same in-house pipeline as above. In total, 17 samples were available, placebo (pre-2, post-3) and treatment (pre-6, post-6), out of which 3 were paired (1-placebo and 2-treatment). The differentially expressed genes between post-treatment and remaining samples resulted in 455 significant genes (fold change of >log₂ 0.75 and an adjusted P-value <0.05).

Flow cytometry. Cells dispersed from cryopreserved tumor tissue or PBMCs were prepared in staining buffer (PBS with 2% FBS and 2 mM EDTA), FcR blocked (clone 2.4G2, BD Biosciences) and stained with antibodies as indicated below for 30 min at 4° C. Cell viability was determined using fixable viability dye (ThermoFisher).

Murine lymphocytes were isolated from the spleen by mechanical dispersion through a 70-μm cell strainer (Miltenyi) to generate single-cell suspensions. RBC lysis (Biolegend) was performed to remove red blood cells. Tumor samples were harvested and lymphocytes were isolated by dispersing the tumor tissue in 2 ml of PBS, followed by incubation of samples at 37° C. for 15 min with DNase I (Sigma) and Liberase DL (Roche). The suspension was then diluted with MACS buffer and passed through a 70-μm cell strainer to generate a single cell suspension. Colons were collected and rinsed in 1 mM dithiothreitol (DTT) to remove feces. Each colon was cut into 2-3 mm pieces and incubated 3 times in pre-digestion solution (HBSS containing 5% FBS and 2 mM EDTA) at 37° C. for 20 min under high rotation to remove epithelial cells. Then tissues were minced with scissors and incubated with digestion solution (HBSS containing 5% FBS, 100 μg/ml DNase I (Sigma) and 1 mg/ml collagenase (Sigma)) at 37° C. for 20 min under high rotation to get single cell solutions of lamina propria cells. Cells were prepared in staining buffer (PBS with 2% FBS and 2 mM EDTA), FcR blocked (clone 2.4G2, BD Biosciences) and stained with antibodies as indicated below for 30 min at 4° C.; secondary stains were done for selected markers. Samples were then sorted or fixed and intracellularly stained using a FoxP3 transcription factor kit according to manufacturer's instructions (eBioscience). Cell viability was determined using fixable viability dye (ThermoFisher). The following antibodies from BD Biosciences, Biolegend, Miltenyi or eBbioscience were used: anti-human PD-1 (EH12.1, 1:30), CD4 (OKT4, 1:30), CD137 (4B4-1, 1:30), GITR (108-17, 1:30), ICOS (C398.4A, 1:50), CD8A (SKI, 1:30), CD25 (M-A251, 1:20), CD3 (SK7, 1:30), CD127 (eBioRDR5, 1:50), CD45 (HI30, 1:30), CD14 (HCD14, 1:50), CD20 (2H7, 1:50); anti-mouse CD3 (145-2C11, 1:100), CD4 (RM4-5, 1:100), CD8 (53-6.7, 1:100), PD-1 (29F1.A12, 1:100), ST2 (U29-93, 1:100) Ki67 (B56, 1:40), TOX (REA473, 1:40), CD19 (6D5, 1:100), CD45 (30-F11, 1:100), FOXP3 (FJK-16s, 1:100), GZMB (QA16A02, 1:40). All samples were acquired on a BD FACS Fortessa or sorted on a BD FACS Fusion (both BD Biosciences) and analyzed using FlowJo 10.4.1 for subsequent single-cell RNA-seq analysis.

Colitis experiments. Dextran Sodium Sulfate, MW ca 40,000 (Alfa Aesar) 2.5% (w/v) was added to the drinking water of mice with ad libitum access. Body weight of the mice was monitored. Colon tissues were collected for histological analysis at the endpoint. Whole colons were harvested from mice between cecum and rectum. Stools were flushed out of lumen with PBS. Then colons were fixed with zinc formalin for 5 minutes. Fixed colons were opened longitudinally, flattened, cut into 3 fragments and further fixed in zinc formalin for 48 hours in cassettes. After fixation, samples were transferred to 70% isopropanol for long term storage or H&E staining. Slides were scored blindly according to the following criteria: inflammation, area of infiltration (extent), crypt damage and edema. The colon was divided into 3 equal parts and the middle section was utilized for scoring. Four randomly selected areas were analyzed, and a histological score was determined.

Single-cell transcriptome analysis. Human. Single-cell RNA-seq was performed by Smart-seq2 as previously described³⁷. Reads were mapped as above. Good quality cells were defined as those with at least 200 genes, at least 60 percent of mapping reads, mitochondrial counts of at most 20%, at least 50,000 total counts (reported by STAR excluding tRNA and rRNA), and a 5′ to 3′ bias of at most 2. Filtered cells were analyzed using the package Seurat (v3.1.5). In order to separate CD4 and CD8 more effectively, differential gene expression analysis was performed between single-positive cells using CD4 and CD8B genes. Cells were clustered using 178 significant genes (adjusted P-value <0.5).

Murine T_{REG} cells. Single-cell RNA-seq was performed by using the 10× platform (10× Genomics, Pleasanton, CA, USA) according to the manufacturer's instructions. Reads were mapped with Cell Ranger followed by QC (github.com/vijaybioinfo/quality_control) and demultiplexed with bcl2fastq using default parameters. Cell Ranger aggr routine was used and CITE-seq data was processed using github.com/vijaybioinfo/ab_capture. Briefly, raw output from Cell Ranger is taken and cell barcodes with less than 100 UMI counts as their top feature are discarded, the remaining barcodes are classified by MULTIseqDemux from Seurat. Finally, cell barcodes where the assigned feature doesn't have the highest UMI count are fixed, and cells with a fold change of less than 3 between the top two features are reclassified as doublets. Before clustering, cells were filtered for at least 300 at most 5000 genes, at least 500 and at most 10000 UMI counts, and at most 5 percent of mitochondrial counts. Cell types were identified using Seurat's FindAllMarkers function. Differential expression was calculated with MAST³⁸ (v1.10.0) DESeq2 (v1.24.0) as previously described³⁷ and genes with an adjusted P-value <0.05 and a fold change of >log₂ 0.5 were defined as significant. P-values were corrected for multiple comparisons using the Benjamini-Hochberg method. GSEA scores were estimated with fgsea (v1.10.1) in R using signal-to-noise ratio as the metric (minSize=3 and maxSize=500). Enrichment scores were shown as GSEA plots. Signature scores were computed using Seurat's AddModuleScore function with default parameters. In short, the score is defined for each cell by subtracting the mean expression of an aggregate of control gene lists from the mean of the signature gene list. Control gene lists were randomly selected (same size as the signature list) from bins delimited based on the level of expression of the signature list.

Murine colonic CD4+ and CD8+ T cells. Single-cell RNA-seq was performed by using the 10× platform. Mapping, aggregation, and QC was carried out as described above with the following thresholds: genes per cells range of [300, 4500], UMI content per cell was [500, 20,000], percent of mitochondrial counts of <=10%, and a doublet score of <=0.3. Cluster of contaminant cells expressing epithelial, monocyte, and fibroblast markers was eliminated after the first round of clustering. The final number of cells were n=6415 CD4⁺ T cells and n=2715 CD8⁺ T cells.

T-cell receptor analysis. TCR were reconstructed from single-cell RNA-seq reads using MiXCR with default parameters. Then, shared TCR were defined by having the same CDR3 sequence in both the alpha and beta chains and coming from the same donor. Enriched TCR were defined as those with a frequency higher or equal to two. Lastly, TCR network plots were generated using the Python package graphviz.

Quantification and statistical analysis. The number of subjects, samples or mice/group, replication in independent experiments, and statistical tests can be found in the figure legends. Details on quality control, sample elimination and displayed data are stated in the methods and figure legends. Sample sizes were chosen based on published studies to ensure sufficient numbers of mice in each group enabling reliable statistical testing and accounting for variability. RNA-seq samples that didn't pass quality control check were excluded from down-stream analyses. Experimental results were reliably reproduced in at least two independent experiments. Animals of same sex and age were randomly assigned to experimental groups. ED FIG. 1A and ED FIG. 3A were created with BioRender.com, the statistical analyses were performed with Graph Pad Prism 9 and statistical tests used are indicated in the figure legends.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Furman, R. R. et al. Idelalisib and Rituximab in Relapsed Chronic Lymphocytic Leukemia. N. Engl. J. Med. (2014) doi:10.1056/nejmoal315226.
2. Gopal, A. K. et al. PI3Kδ Inhibition by Idelalisib in Patients with Relapsed Indolent Lymphoma. N. Engl. J. Med. 370:997-1007 (2014).
3. Vanhaesebroeck, B., Perry, M. W. D., Brown, J. R., André, F. & Okkenhaug, K. PI3K inhibitors are finally coming of age. Nat. Rev. Drug Discov. 2021 2010 20, 741-769 (2021).
4. Ali, K. et al. Inactivation of PI(3)K p110δ breaks regulatory T-cell-mediated immune tolerance to cancer. Nature 510, 407-411 (2014).
5. Lim, E. L. et al. Phosphoinositide 3-kinase δ inhibition promotes antitumor responses but antagonizes checkpoint inhibitors. JCI insight 3(11):e120626 (2018).
6. Lampson, B. L. et al. Idelalisib given front-line for treatment of chronic lymphocytic leukemia causes frequent immune-mediated hepatotoxicity. Blood 128(2):195-203 (2016).
7. Lim, E. L. & Okkenhaug, K. Phosphoinositide 3-kinase δ is a regulatory T-cell target in cancer immunotherapy. Immunology 157(3):210-218 (2019).
8. Chellappa, S. et al. The PI3K p110δ Isoform Inhibitor Idelalisib Preferentially Inhibits Human Regulatory T Cell Function. J. Immunol. 202(5):1397-1405 (2019).
9. Scott, A. C. et al. TOX is a critical regulator of tumour-specific T cell differentiation. Nature 571(7764):270-274 (2019).
10. Khan, O. et al. TOX transcriptionally and epigenetically programs CD8+ T cell exhaustion. Nature 571(7764):211-218 (2019).
11. Alfei, F. et al. TOX reinforces the phenotype and longevity of exhausted T cells in chronic viral infection. Nature 571(7764):265-269 (2019).
12. Lampson, B. L. & Brown, J. R. PI3Kδ-selective and PI3Kα/δ-combinatorial inhibitors in clinical development for B-cell non-Hodgkin lymphoma. Expert Opinion on Investigational Drugs 26(11):1267-1279 (2017).
13. DiSpirito, J. R. et al. Molecular diversification of regulatory T cells in nonlymphoid tissues. Sci. Immunol. 3(27), eaat5861 (2018).
14. Miragaia, R. J. et al. Single-Cell Transcriptomics of Regulatory T Cells Reveals Trajectories of Tissue Adaptation. Immunity 50(2):493-504.e7 (2019).
15. Wing, J. B., Ise, W., Kurosaki, T. & Sakaguchi, S. Regulatory T cells control antigen-specific expansion of Tfh cell number and humoral immune responses via the coreceptor CTLA-4. Immunity 41, 1013-1025 (2014).
16. Ovcinnikovs, V. et al. CTLA-4-mediated transendocytosis of costimulatory molecules primarily targets migratory dendritic cells. Sci. Immunol. 4(35):eeaw0902 (2019).
17. Zhang, N. et al. Regulatory T Cells Sequentially Migrate from Inflamed Tissues to Draining Lymph Nodes to Suppress the Alloimmune Response. Immunity 30(3):458-69 (2009).
18. Delacher, M. et al. Precursors for Nonlymphoid-Tissue Treg Cells Reside in Secondary Lymphoid Organs and Are Programmed by the Transcription Factor BATF. Immunity 52(2):295-312 (2020).
19. Whibley, N., Tucci, A. & Powrie, F. Regulatory T cell adaptation in the intestine and skin. Nature Immunology 20(4):386-396 (2019).
20. Arpaia, N. et al. A Distinct Function of Regulatory T Cells in Tissue Protection. Cell 162(5):1078-89 (2015).
21. Aaronson, D. S. & Horvath, C. M. A road map for those who don't know JAK-STAT. Science 296(5573):1653-5 (2002).
22. Hu, X. & Ivashkiv, L. B. Cross-regulation of Signaling Pathways by Interferon-y: Implications for Immune Responses and Autoimmune Diseases. Immunity 31(4):539-50 (2009).
23. La Manno, G. et al. RNA velocity of single cells. Nature 560, 494-498 (2018).
24. Bergen, V., Lange, M., Peidli, S., Wolf, F. A. & Theis, F. J. Generalizing RNA velocity to transient cell states through dynamical modeling. Nat. Biotechnol. 2020 3812 38, 1408-1414 (2020).
25. L, R. et al. Differential regulation of interleukin 17 and interferon gamma production in inflammatory bowel disease. Gut 58, 1629-1636 (2009).
26. Harbour, S. N., Maynard, C. L., Zindl, C. L., Schoeb, T. R. & Weaver, C. T. Th17 cells give rise to Th1 cells that are required for the pathogenesis of colitis. Proc. Natl. Acad. Sci. 112, 7061-7066 (2015).
27. Feng, T. et al. Th17 Cells Induce Colitis and Promote Th1 Cell Responses through IL-17 Induction of Innate IL-12 and IL-23 Production. J. Immunol. 186, 6313-6318 (2011).
28. Denning, T. L. et al. Mouse TCRαβ+CD8αα Intraepithelial Lymphocytes Express Genes That Down-Regulate Their Antigen Reactivity and Suppress Immune Responses. J. Immunol. 178, 4230-4239 (2007).
29. Poussier, P., Ning, T., Banerjee, D. & Julius, M. A Unique Subset of Self-specific Intraintestinal T Cells Maintains Gut Integrity. J. Exp. Med. 195, 1491-1497 (2002).
30. Chaudhry, A. et al. CD4+ regulatory T cells control TH17 responses in a stat3-dependent manner. Science 326, 986-991 (2009).
31. Ward, M. J. et al. Staging and treatment of oropharyngeal cancer in the human papillomavirus era. Head Neck 37(7):1002-13 (2015).
32. Wood, O. et al. Gene expression analysis of TIL rich HPV-driven head and neck tumors reveals a distinct B-cell signature when compared to HPV independent tumors. Oncotarget 7, 56781-56797 (2016).
33. Ottensmeier, C. H. et al. Upregulated Glucose Metabolism Correlates Inversely with CD8+T-cell Infiltration and Survival in Squamous Cell Carcinoma. Cancer Res. 76, 4136-4148 (2016).
34. Lanasa, M. C. et al. First-In-Human Study Of AMG 319, a Highly Selective, Small Molecule Inhibitor Of PI3KS, In Adult Patients With Relapsed Or Refractory Lymphoid Malignancies. Blood (2013) 122(21):678.
35. Vijayanand, P. et al. Invariant Natural Killer T Cells in Asthma and Chronic Obstructive Pulmonary Disease. N. Engl. J. Med. 356(14):1410-22 (2007).
36. Juneja, V. R. et al. PD-L1 on tumor cells is sufficient for immune evasion in immunogenic tumors and inhibits CD8 T cell cytotoxicity. J. Exp. Med. 214, 895-904 (2017).
37. Patil, V. S. et al. Precursors of human CD4+cytotoxic T lymphocytes identified by single-cell transcriptome analysis. Sci. Immunol. 3(19), 8664 (2018).
38. Finak, G. et al. MAST: A flexible statistical framework for assessing transcriptional changes and characterizing heterogeneity in single-cell RNA sequencing data. Genome Biol. 16, 278 (2015).

What is claimed is:

1. A method of treating cancer in a patient, the method comprising the steps of:
(a) providing or obtaining a sample from the patient;
(b) determining at least one of a level or activity of at least one of: T follicular regulatory (T_{FR}), ST2 Treg, highly suppressive Treg, activated Treg, or effector Treg cells in the sample;
(c) comparing the at least one of level or activity of the at least one of T_{FR} cells, ST2 Treg, highly suppressive Treg, activated Treg, or effector Treg in the sample to a level or activity of T_{FR}, ST2 Treg, highly suppressive Treg, activated Treg, or effector Treg cells in a reference sample, respectively, for a specific tumor type or a healthy subject; and
(d) if the patient has a high level or activity of T_{FR}, ST2 Treg, highly suppressive Treg, activated Treg, or effector Treg cells systemically as compared to the reference sample, then administering a cancer therapy to the patient that comprises a modified dosage or administration of a Phosphoinositide 3-kinase (PI3K) inhibitor.

2. The method of claim 1, wherein the modified dosage or administration of the PI3K inhibitor selectively depletes T_{FR}, ST2 Treg, highly suppressive Treg, activated Treg, or effector Treg cells preferentially in a tumor microenvironment, in tumor-draining lymph nodes, or both.

3. The method of claim 1, wherein the modified dosage or administration of the PI3K inhibitor transiently depletes T_{FR}, ST2 Treg, highly suppressive Treg, activated Treg, or effector Treg cells systemically.

4. The method of claim 1, wherein the modified dosage or administration of the PI3K inhibitor preferentially depletes T_{FR}, ST2 Treg, highly suppressive Treg, activated Treg, or effector Treg cells in a tumor microenvironment.

5. The method of any one of claims 1 to 4, wherein the PI3K inhibitor is provided in conjunction with, or followed by, an additional cancer therapy.

6. The method of claim 5, wherein the additional cancer therapy is a checkpoint inhibitor or other immunotherapy.

7. The method of claim 6, wherein the immune checkpoint inhibitor or immunotherapy comprises an antibody directed against a cellular protein selected from at least one of: PD-1, PD-L1, CTLA-4, TIM-3, BTLA, LAG-3, or TIGIT.

8. The method of claim 1, wherein the PI3K inhibitor is a selective Phosphoinositide 3-kinase δ inhibitor.

9. The method of claim 8, wherein the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114.

10. The method of claim 1, wherein the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺GITR⁺ T cells, or any combination thereof.

11. The method of claim 1, wherein the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1.

12. The method of claim 1, wherein the method further comprises isolating the T_{FR} cells from the sample prior to determining the level or activity of T_{FR} cells in the sample.

13. The method of claim 1, wherein the cancer is selected from a colorectal, a melanoma, a lung, a liver, a head and neck, and a breast cancer.

14. The method of claim 1, wherein the sample is a tumor biopsy.

15. The method of claim 1, wherein the step of determining the level or activity of T_{FR}, ST2 Treg, highly suppressive Treg, activated Treg, or effector Treg cells in the sample is performed by measuring mRNA, protein, or both.

16. A method of stratifying cancer patients to select an effective cancer treatment for administration, comprising:
(a) providing or obtaining a sample from a patient;
(b) determining at least one of a level or activity of T-follicular regulatory (T_{FR}), ST2 Treg, highly suppressive Treg, activated Treg, or effector Treg cells in the sample;
(c) comparing the at least one of a level or activity of T_{FR} cells in the sample to a level or activity of T_{FR}, ST2 Treg, highly suppressive Treg, activated Treg, or effector Treg cells in a reference sample, respectively, for a specific tumor type or a healthy subject; and
(d) stratifying the patient into at least one of three groups selected from: (1) a high level or increase in the level or activity of T_{FR}, ST2 Treg, highly suppressive Treg, activated Treg, or effector Treg cells in a tumor microenvironment or tumor-draining lymph nodes, (2) a high level or activity of T_{FR}, ST2 Treg, highly suppressive Treg, activated Treg, or effector Treg cells systemically, or (3) a low level or activity of T_{FR}, ST2 Treg, highly suppressive Treg, activated Treg, or effector Treg cells in a tumor microenvironment or tumor-draining lymph node, or (4) a low level or activity of T_{FR}, ST2 Treg, highly suppressive Treg, activated Treg, or effector Treg cells systemically, when compared to the reference for the specific tumor type or the healthy subject.

17. The method of claim 16, wherein if the patient has a high level or activity of T_{FR}, ST2 Treg, highly suppressive Treg, activated Treg, or effector Treg cells systemically, then selecting a cancer treatment that does not consist of administration of a Phosphoinositide 3-kinase (PI3K) inhibitor.

18. The method of claim 16, wherein if the patient has an increase in the level or activity of T_{FR}, ST2 Treg, highly suppressive Treg, activated Treg, or effector Treg cells in the tumor microenvironment or tumor-draining lymph nodes, further comprising administering a T_{FR} cell depleting therapy capable of selectively depleting T_{FR}, ST2 Treg, highly suppressive Treg, activated Treg, or effector Treg cells preferentially in the tumor microenvironment or tumor-draining lymph nodes, or both, prior to or concurrent with administration of a cancer treatment.

19. The method of claim 16, wherein if the patient has a high level or an increase in the level or activity of T_{FR}, ST2 Treg, highly suppressive Treg, activated Treg, or effector Treg cells in the tumor microenvironment or tumor-draining lymph nodes or a high level or activity of T_{FR}, ST2 Treg, highly suppressive Treg, activated Treg, or effector Treg cells systemically, further comprising administering a T_{FR} cell depleting therapy capable of transiently depleting T_{FR}, ST2 Treg, highly suppressive Treg, activated Treg, or effector Treg cells systemically prior to or concurrent with administration of a cancer treatment.

20. The method of claim 16, wherein if the patient has a high level or an increase in the level or activity of T_{FR}, ST2 Treg, highly suppressive Treg, activated Treg, or effector Treg cells in the tumor microenvironment or tumor-draining lymph nodes, further comprising administering a T_{FR}, ST2 Treg, highly suppressive Treg, activated Treg, or effector Treg cell depleting therapy capable of preferentially depleting T_{FR}, ST2 Treg, highly suppressive Treg, activated Treg, or effector Treg cells in the tumor microenvironment or tumor-draining lymph nodes prior to or concurrent with administration of a cancer treatment.

21. The method of claim 16, wherein if the patient has a low level or decrease or low activity of T_{FR}, ST2 Treg, highly suppressive Treg, activated Treg, or effector Treg cells in the tumor microenvironment or tumor-draining lymph node, further comprising administering an immunotherapy agent or antibody.

22. The method of claim 16, wherein if the patient has a low level or decrease or low activity of T_{FR}, ST2 Treg, highly suppressive Treg, activated Treg, or effector Treg cells systemically, further comprising administering an immunotherapy agent or antibody.

23. The method of any one of claims 18-20, wherein the T_{FR}, ST2 Treg, highly suppressive Treg, activated Treg, or effector Treg cell depleting therapy is provided in conjunction with, or followed by, the cancer treatment.

24. The method of any one of claims 16-20, 23, wherein the cancer treatment is a checkpoint inhibitor or other immunotherapy.

25. The method of claim 21-23, wherein the checkpoint inhibitor or immunotherapy comprises an antibody directed against a cellular protein selected from at least one of: PD-1, PD-L1, CTLA-4, TIM-3, BTLA, LAG-3, or TIGIT.

26. The method of any one of claims 18-25, wherein the T_{FR} cell depleting therapy comprises administering one or more of the following: a modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on TFR cells when compared to T_{REG} cells and other T cell populations, wherein the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off.

27. The method of any one of claims 18-26, wherein the T_{FR} cell depleting therapy does not substantially reduce or eliminate T_{REGS}.

28. The method of claim 26, wherein the PI3K inhibitor is a selective Phosphoinositide 3-kinase δ inhibitor.

29. The method of claim 28, wherein the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114.

30. The method of claim 16, wherein the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺ GITR⁺ T cells, or any combination thereof.

31. The method of claim 16, wherein the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1.

32. The method of claim 16, wherein the cancer is selected from a colorectal, a melanoma, a lung, a liver, a head and neck, and a breast cancer.

33. The method of claim 16, wherein the patient sample is a tumor biopsy.

34. The method of claim 16, wherein the step of determining the level or activity of T follicular regulatory (T_{FR}), ST2 Treg, highly suppressive Treg, activated Treg, or effector Treg cells in the sample is performed by measuring mRNA, protein, or both.

35. A method of treating cancer, comprising:
(a) providing or obtaining a sample from a patient;
(b) determining at least one of a level or activity of at least one of: T-follicular regulatory (T_{FR}), ST2 Treg, highly suppressive Treg, activated Treg, or effector Treg cells in the sample;
(c) comparing the least one of level or activity of T_{FR} cells in the sample to a level or activity of TFR cells in a reference sample, respectively, for a specific tumor type or a healthy subject;
(d) stratifying the patient into at least one of three groups selected from: (1) an increase in the level or activity of T_{FR}, ST2 Treg, highly suppressive Treg, activated Treg, or effector Treg cells in a tumor microenvironment or tumor-draining lymph nodes, (2) a high level or activity of T_{FR}, ST2 Treg, highly suppressive Treg, activated Treg, or effector Treg cells systemically, or (3) a low level or activity of T_{FR}, ST2 Treg, highly suppressive Treg, activated Treg, or effector Treg cells in a tumor microenvironment or tumor-draining lymph node, or (4) a low level or activity of T_{FR}, ST2 Treg, highly suppressive Treg, activated Treg, or effector Treg cells systemically, when compared to the reference for the specific tumor type or the healthy subject; and
(e) administering an appropriate cancer treatment based on the stratification step of (d).

36. The method of claim 35, wherein if the patient has a high level or activity of T_{FR}, ST2 Treg, highly suppressive Treg, activated Treg, or effector Treg cells systemically, then selecting a cancer treatment that does not consist of administration of a Phosphoinositide 3-kinase (PI3K) inhibitor.

37. The method of claim 35, wherein if the patient has an increase in the level or activity of T_{FR} cells in a tumor microenvironment or tumor-draining lymph nodes, further comprising administering a T_{FR}, ST2 Treg, highly suppressive Treg, activated Treg, or effector Treg cell depleting therapy capable of selectively depleting T_{FR}, ST2 Treg, highly suppressive Treg, activated Treg, or effector Treg cells preferentially in the tumor microenvironment or tumor-draining lymph nodes, or both, prior to or concurrent with the administration of the cancer treatment.

38. The method of claim 35, wherein if the patient has an increase in the level or activity of T_{FR}, ST2 Treg, highly suppressive Treg, activated Treg, or effector Treg cells in a tumor microenvironment or tumor-draining lymph nodes or a high level or activity of T_{FR}, ST2 Treg, highly suppressive Treg, activated Treg, or effector Treg cells systemically, further comprising administering a T_{FR}, ST2 Treg, highly suppressive Treg, activated Treg, or effector Treg cell depleting therapy capable of transiently depleting T_{FR} cells systemically prior to or concurrent with the administration of the cancer treatment.

39. The method of claim 35, wherein if the patient has an increase in the level or activity of T_{FR}, ST2 Treg, highly suppressive Treg, activated Treg, or effector Treg cells in a tumor microenvironment or tumor-draining lymph nodes, further comprising administering a T_{FR}, ST2 Treg, highly suppressive Treg, activated Treg, or effector Treg cell depleting therapy capable of preferentially depleting T_{FR}, ST2 Treg, highly suppressive Treg, activated Treg, or effector Treg cells in the tumor microenvironment or tumor-draining lymph nodes prior to or concurrent with the administration of the cancer treatment.

40. The method of claim 35, wherein if the patient has a low level or decrease or low activity of T_{FR}, ST2 Treg, highly suppressive Treg, activated Treg, or effector Treg cells in a tumor microenvironment or tumor-draining lymph node, further comprising administering an immunotherapy agent or antibody.

41. The method of claim 35, wherein if the patient has a low level or decrease or low activity of T_{FR}, ST2 Treg, highly suppressive Treg, activated Treg, or effector Treg cells systemically, further comprising administering an immunotherapy agent or antibody.

42. The method of any one of claims 35-39, wherein the T_{FR}, ST2 Treg, highly suppressive Treg, activated Treg, or effector Treg cell depleting therapy is provided in conjunction with, or followed by, the cancer treatment.

43. The method of any one of claims 35-42, wherein the cancer treatment is a checkpoint inhibitor or other immunotherapy agent or antibody.

44. The method of claim 43, wherein the checkpoint inhibitor or immunotherapy agent comprises an antibody directed against a cellular protein selected from at least one of: PD-1, PD-L1, CTLA-4, TIM-3, BTLA, LAG-3, or TIGIT.

45. The method of claim 40 or 41 wherein the immunotherapy agent comprises an antibody directed against a cellular protein selected from at least one of: PD-1, PD-L1, TIM-3, BTLA, LAG-3, or TIGIT.

46. The method of any one of claims 35-39, or 42-44, wherein the T_{FR} cell depleting therapy comprises administering one or more of the following: a modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on T_{FR} cells when compared to T_{REG} cells and other T cell populations, wherein the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off.

47. The method of any one of claims 35-46, wherein the T_{FR} cell depleting therapy does not substantially reduce or eliminate T_{REGS}.

48. The method of claim 46, wherein the PI3K inhibitor is a selective Phosphoinositide 3-kinase δ inhibitor.

49. The method of claim 48, wherein the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114.

50. The method of claim 35, wherein the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺GITR⁺ T cells, or any combination thereof.

51. The method of claim 35, wherein the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1.

52. The method of claim 35, wherein the cancer is selected from a colorectal, a melanoma, a lung, a liver, a head and neck, and a breast cancer.

53. The method of claim 35, wherein the patient sample is a tumor biopsy.

54. The method of claim 35, wherein the step of determining the level or activity of T follicular regulatory (T_{FR}) cells in the sample is performed by measuring mRNA, protein, or both.

55. A method of treating a patient with a cancer vaccine, the method comprising the steps of:
(a) providing or obtaining a sample from the patient;
(b) determining at least one of a level or activity of at least one of: T follicular regulatory (T_{FR}), ST2 Treg, highly suppressive Treg, activated Treg, or effector Treg cells in the sample;
(c) comparing the at least one of level or activity of T_{FR}, ST2 Treg, highly suppressive Treg, activated Treg, or effector Treg cells in the sample to a level or activity of T_{FR} cells in a reference sample, respectively, for a specific tumor type or a healthy subject; and
(d) if the patient has a high level or activity of T_{FR}, ST2 Treg, highly suppressive Treg, activated Treg, or effector Treg cells systemically as compared to the reference sample, then administering a modified dosage or administration of an agent that reduces the level or activity of T_{FR}, ST2 Treg, highly suppressive Treg, activated Treg, or effector Treg cells systemically prior to, or concurrent with, administration of a cancer vaccine to the patient.

56. The method of claim 55, wherein the cancer vaccine is a tumor cell vaccine, an antigen vaccine, a dendritic cell vaccine, a DNA vaccine, an mRNA vaccine or a vector-based vaccine.

57. The method of claim 55, wherein the cancer vaccine is directed to a cancer antigen selected from at least one of: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, NY-ESO-1, LAGE-1, SSX-1, SSX-2 (HOM-MEL-40), SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RARalpha fusion protein, PTPRK, K-ras, N-ras, Triosephosphate isomerase, GnTV, Herv-K-mel, Lage-1, NA-88, /Lage-2, SP17, and TRP2-Int2, (MART-I), gp100, TRP-1, TRP-2, MAGE-1, MAGE-3, pi5(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, pi85erbB2, pi80erbB-3, c-met, nm-23H1, PSA, TAG-72-4, CA 19-9, CA 72-4, CAM 17.1, NuMa, K-ras, CDK4, Mum-1, p16, TAGE, PSMA, PSCA, CT7, telomerase, 43-9F, 5T4, 791Tgp72, alpha-fetoprotein, 13HCG, BCA225, BTAA, CA 125, CA 15-3, CA 195, CA 242, CA-50, CAM43, CD68\KP1, CO-029, FGF-5, G250, Ga733, HTgp-175, M344, MA-50, MG7-Ag, MOV18, NB\170K, NY-CO-1, RCAS1, SDCCAG16, TA-90, TAAL6, TAG72, TLP, TPS; or immunogenic fragments that comprise an epitope of any of the foregoing antigens.

58. The method of claim 55, wherein the agent comprises a modified dosage or administration of a PI3K inhibitor that selectively or transiently depletes T_{FR}, ST2 Treg, highly suppressive Treg, activated Treg, or effector Treg cells preferentially in a tumor microenvironment, in tumor-draining lymph nodes, or systemically.

59. The method of claim 55, wherein the PI3K inhibitor is provided in conjunction with, or followed by, an additional cancer therapy.

60. The method of claim 59, wherein the additional cancer therapy is a checkpoint inhibitor or other immunotherapy.

61. The method of claim 60, wherein the immune checkpoint inhibitor or immunotherapy comprises an antibody directed against a cellular protein selected from at least one of: PD-1, PD-L1, CTLA-4, TIM-3, BTLA, LAG-3, or TIGIT.

62. The method of claim 55, wherein the T_{FR} cell depleting therapy comprises administering one or more of the following a modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on T_{FR} cells when compared to T_{REG} cells and other T cell populations, wherein the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off.

63. The method of claim 59, wherein the PI3K inhibitor is a selective Phosphoinositide 3-kinase δ inhibitor.

64. The method of claim 63, wherein the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114.

65. The method of claim 55, wherein the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺ GITR⁺ T cells, or any combination thereof.

66. The method of claim 55, wherein the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1.

67. The method of claim 55, wherein the method further comprises isolating the T_{FR}, ST2 Treg, highly suppressive Treg, activated Treg, or effector Treg cells from the sample prior to determining the level or activity of T_{FR} cells in the sample.

68. The method of claim 55, wherein the cancer is selected from a colorectal, a melanoma, a lung, a liver, a head and neck, and a breast cancer.

69. The method of claim 55, wherein the sample is a tumor biopsy.

70. The method of claim 55, wherein the step of determining the level or activity of T_{FR}, ST2 Treg, highly suppressive Treg, activated Treg, or effector Treg cells in the sample is performed by measuring mRNA, protein, or both.

71. A method of stratifying cancer patients for administration of a cancer vaccine, comprising:
(a) providing or obtaining a sample from a patient;
(b) determining at least one of a level or activity of T-follicular regulatory (T_{FR}), ST2 Treg, highly suppressive Treg, activated Treg, or effector Treg cells in the sample;
(c) comparing the at least one of a level or activity of T_{FR}, ST2 Treg, highly suppressive Treg, activated Treg, or effector Treg cells in the sample to a level or activity of T_{FR} cells in a reference sample, respectively, for a specific tumor type or a healthy subject; and
(d) stratifying the patient into at least one of three groups selected from: (1) a high level or increase in the level or activity of T_{FR}, ST2 Treg, highly suppressive Treg, activated Treg, or effector Treg cells in a tumor microenvironment or tumor-draining lymph nodes, (2) a high level or activity of T_{FR}, ST2 Treg, highly suppressive Treg, activated Treg, or effector Treg cells systemically, or (3) a low level or activity of T_{FR}, ST2 Treg, highly suppressive Treg, activated Treg, or effector Treg cells in a tumor microenvironment or tumor-draining lymph node, or (4) a low level or activity of T_{FR}, ST2 Treg, highly suppressive Treg, activated Treg, or effector Treg cells systemically, when compared to the reference for the specific tumor type or the healthy subject, wherein the cancer vaccine is administered to the patient.

72. The method of claim 71, wherein the cancer vaccine is a tumor cell vaccine, an antigen vaccine, a dendritic cell vaccine, a DNA vaccine, an mRNA vaccine or a vector-based vaccine.

73. The method of claim 71, wherein if the patient is in groups (1) or (2), the patient is also provided with a T_{FR} cell depleting therapy.

74. The method of claim 71, wherein the cancer vaccine is directed to a cancer antigen selected from at least one of: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, NY-ESO-1, LAGE-1, SSX-1, SSX-2 (HOM-MEL-40), SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RARalpha fusion protein, PTPRK, K-ras, N-ras, Triosephosphate isomerase, GnTV, Herv-K-mel, Lage-1, NA-88, /Lage-2, SP17, and TRP2-Int2, (MART-I), gp100, TRP-1, TRP-2, MAGE-1, MAGE-3, p15(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, mn-23H1, PSA, TAG-72-4, CA 19-9, CA 72-4, CAM 17.1, NuMa, K-ras, CDK4, Mum-1, p16, TAGE, PSMA, PSCA, CT7, telomerase, 43-9F, 5T4, 791Tgp72, alpha-fetoprotein, 13HCG, BCA225, BTAA, CA 125, CA 15-3, CA 195, CA 242, CA-50, CAM43, CD68\KP1, CO-029, FGF-5, G250, Ga733, HTgp-175, M344, MA-50, MG7-Ag, MOV18, NB\170K, NY-CO-1, RCAS1, SDCCAG16, TA-90, TAAL6, TAG72, TLP, TPS; or immunogenic fragments that comprise an epitope of any of the foregoing antigens.

75. The method of claim 71, wherein if the patient has an increase in the level or activity of T_{FR}, ST2 Treg, highly suppressive Treg, activated Treg, or effector Treg cells in the tumor microenvironment or tumor-draining lymph nodes, further comprising administering a T_{FR}, ST2 Treg, highly suppressive Treg, activated Treg, or effector Treg cell depleting therapy capable of selectively or transiently depleting T_{FR}, ST2 Treg, highly suppressive Treg, activated Treg, or effector Treg cells preferentially in the tumor microenvironment or tumor-draining lymph nodes, or both, prior to or concurrent with, administration of a cancer vaccine.

76. The method of claim 71, wherein if the patient has a high level or an increase in the level or activity of T_{FR}, ST2 Treg, highly suppressive Treg, activated Treg, or effector Treg cells in the tumor microenvironment or tumor-draining lymph nodes, further comprising administering a T_{FR}, ST2 Treg, highly suppressive Treg, activated Treg, or effector Treg cell depleting therapy capable of preferentially depleting T_{FR} cells in the tumor microenvironment or tumor-draining lymph nodes prior to or concurrent with administration of a cancer vaccine.

77. The method of claim 71, wherein if the patient has a low level or decrease or low activity of T_{FR}, ST2 Treg, highly suppressive Treg, activated Treg, or effector Treg cells in the tumor microenvironment or tumor-draining lymph node, further comprising administering an immunotherapy agent or antibody.

78. The method of claim 71, wherein if the patient has a low level or decrease or low activity of TFR cells systemically, further comprising administering an immunotherapy agent or antibody.

79. The method of any one of claim 75 or 76, wherein the T_{FR} cell depleting therapy is provided in conjunction with, or followed by, a cancer treatment.

80. The method of any one of claim 78 or 79, wherein the cancer treatment is a checkpoint inhibitor or other immunotherapy.

81. The method of any one of claims 77 to 80, wherein the checkpoint inhibitor or immunotherapy comprises an antibody directed against a cellular protein selected from at least one of: PD-1, PD-L1, CTLA-4, TIM-3, BTLA, LAG-3, or TIGIT.

82. The method of any one of claims 75 to 81, wherein the T_{FR} cell depleting therapy comprises administering one or more of the following: a modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on TFR cells when compared to T_{REG} cells and other T cell populations, wherein the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off.

83. The method of any one of claims 75 to 82, wherein the T_{FR}, ST2 Treg, highly suppressive Treg, activated Treg, or effector Treg cell depleting therapy does not substantially reduce or eliminate T_{REGS}.

84. The method of claim 82, wherein the PI3K inhibitor is a selective Phosphoinositide 3-kinase δ inhibitor.

85. The method of claim 84, wherein the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114.

86. The method of claim 71, wherein the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺ GITR⁺ T cells, or any combination thereof.

87. The method of claim 71, wherein the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1.

88. The method of claim 71, wherein the cancer is selected from a colorectal, a melanoma, a lung, a liver, a head and neck, and a breast cancer.

89. The method of claim 71, wherein the patient sample is a tumor biopsy.

90. The method of claim 71, wherein the step of determining the level or activity of T follicular regulatory (T_{FR}) cells in the sample is performed by measuring mRNA, protein, or both.

91. A method of treating cancer with a cancer vaccine, comprising:
(a) providing or obtaining a sample from a patient;
(b) determining at least one of a level or activity of at least one of: T-follicular regulatory (T_{FR}), ST2 Treg, highly suppressive Treg, activated Treg, or effector Treg cells in the sample;
(c) comparing the least one of level or activity of T_{FR}, ST2 Treg, highly suppressive Treg, activated Treg, or effector Treg cells in the sample to a level or activity of T_{FR} cells in a reference sample, respectively, for a specific tumor type or a healthy subject;
(d) stratifying the patient into at least one of three groups selected from: (1) an increase in the level or activity of T_{FR}, ST2 Treg, highly suppressive Treg, activated Treg, or effector Treg cells in a tumor microenvironment or tumor-draining lymph nodes, (2) a high level or activity of T_{FR}, ST2 Treg, highly suppressive Treg, activated Treg, or effector Treg cells systemically, or (3) a low level or activity of T_{FR}, ST2 Treg, highly suppressive Treg, activated Treg, or effector Treg cells in a tumor microenvironment or tumor-draining lymph node, or (4) a low level or activity of T_{FR}, ST2 Treg, highly suppressive Treg, activated Treg, or effector Treg cells systemically, when compared to the reference for the specific tumor type or the healthy subject; and
(e) administering the cancer vaccine based on the stratification step of (d).

92. The method of claim 91, wherein if the patient has a high level or activity of T_{FR}, ST2 Treg, highly suppressive Treg, activated Treg, or effector Treg cells systemically, then selecting a cancer vaccine that does not require treatment with a Phosphoinositide 3-kinase (PI3K) inhibitor.

93. The method of claim 91, wherein if the patient has an increase in the level or activity of T_{FR}, ST2 Treg, highly suppressive Treg, activated Treg, or effector Treg cells in a tumor microenvironment or tumor-draining lymph nodes, further comprising administering a T_{FR}, ST2 Treg, highly suppressive Treg, activated Treg, or effector Treg cell depleting therapy capable of selectively or transiently depleting T_{FR}, ST2 Treg, highly suppressive Treg, activated Treg, or effector Treg cells preferentially in the tumor microenvironment or tumor-draining lymph nodes, or both, prior to or concurrent with the administration of the cancer vaccine.

94. The method of claim 91, wherein if the patient has an increase in the level or activity of T_{FR}, ST2 Treg, highly suppressive Treg, activated Treg, or effector Treg cells in a tumor microenvironment or tumor-draining lymph nodes or a high level or activity of T_{FR}, ST2 Treg, highly suppressive Treg, activated Treg, or effector Treg cells systemically, further comprising administering a T_{FR}, ST2 Treg, highly suppressive Treg, activated Treg, or effector Treg cell depleting therapy capable of transiently depleting T_{FR}, ST2 Treg, highly suppressive Treg, activated Treg, or effector Treg cells systemically prior to or concurrent with the administration of the cancer vaccine.

95. The method of claim 91, wherein if the patient has an increase in the level or activity of T_{FR}, ST2 Treg, highly suppressive Treg, activated Treg, or effector Treg cells in a tumor microenvironment or tumor-draining lymph nodes, further comprising administering a T_{FR}, ST2 Treg, highly suppressive Treg, activated Treg, or effector Treg cell depleting therapy capable of preferentially or transiently depleting T_{FR} cells in the tumor microenvironment or tumor-draining lymph nodes prior to or concurrent with the administration of the cancer vaccine.

96. The method of claim 91, wherein if the patient has a low level or decrease or low activity of T_{FR}, ST2 Treg, highly suppressive Treg, activated Treg, or effector Treg cells in a tumor microenvironment or tumor-draining lymph node, further comprising administering an immunotherapy agent or antibody.

97. The method of claim 91, wherein if the patient has a low level or decrease or low activity of T_{FR}, ST2 Treg, highly suppressive Treg, activated Treg, or effector Treg cells systemically, further comprising administering an immunotherapy agent or antibody.

98. The method of any one of claims 91 to 94, wherein the T_{FR}, ST2 Treg, highly suppressive Treg, activated Treg, or effector Treg cell depleting therapy is provided in conjunction with, or followed by, the cancer vaccine.

99. The method of any one of claims 91 to 98, further comprising providing a cancer treatment selected from a checkpoint inhibitor or other immunotherapy agent or antibody.

100. The method of claim 99, wherein the checkpoint inhibitor or immunotherapy agent comprises an antibody directed against a cellular protein selected from at least one of: PD-1, PD-L1, CTLA-4, TIM-3, BTLA, LAG-3, or TIGIT.

101. The method of any one of claim 96 or 97, wherein the immunotherapy agent comprises an antibody directed against a cellular protein selected from at least one of: PD-1, PD-L1, TIM-3, BTLA, LAG-3, or TIGIT.

102. The method of any one of claims 91 to 94, or 99 to 101, wherein the TFR cell depleting therapy comprises administering one or more of the following: a modified dose or intermittent administration schedule of a PI3K inhibitor, anti-IL1R2, anti-CTLA-4, anti-TIGIT, anti-4-1BB, anti-ICOS, anti-GITR, anti-OX40, anti-TNFR2, or anti-CCR8 therapy, or other cell surface targets specifically expressed or enriched on T_{FR} cells when compared to T_{REG} cells and other T cell populations, wherein the intermittent administration schedule is 1 day on the PI3K inhibitor followed by 2, 3, 4, 5, 6 or 7 days off the PI3K inhibitor, 2 days on followed by 2, 3, 4, 5, 6 or 7 days off; 3 days on followed by 2, 3, 4, 5, 6 or 7 days off drug, 4 days on followed by 2, 3, 4, 5, 6 or 7 days off, or 5 days on followed by 2, 3, 4, 5, 6 or 7 days off.

103. The method of any one of claims 91 to 102, wherein the T_{FR}, ST2 Treg, highly suppressive Treg, activated Treg, or effector Treg cell depleting therapy does not substantially reduce or eliminate T_{REGS}.

104. The method of claim 102, wherein the PI3K inhibitor is a selective Phosphoinositide 3-kinase δ inhibitor.

105. The method of claim 104, wherein the selective Phosphoinositide 3-kinase δ inhibitor is selected from at least one of: AMG319, CAL-101 (Idelalisib, GS-1101), PIK-294, PI-3065, PIK-293, Zandelisib, IOA-244, Zydelig, Aliqupa, Ukoniq, or IC-87114.

106. The method of claim 91, wherein the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺ GITR⁺ T cells, or any combination thereof.

107. The method of claim 91, wherein the T_{FR} cells express one or more of the following markers: FOXP3, GITR, CXCR5, BCL-6, CTLA-4, 4-1BB, ICOS, TOX, KI-67, TCF-1, TNFRSF1B (TNFR2), LAG-3, TIGIT, BATF, IL1R2, CCR8, PD-1, TOX, DUSP14, or CLP1.

108. The method of claim 91, wherein the cancer is selected from a colorectal, a melanoma, a lung, a liver, a head and neck, and a breast cancer.

109. The method of claim 91, wherein the patient sample is a tumor biopsy.

110. The method of claim 91, wherein the step of determining the level or activity of T follicular regulatory (T_{FR}), ST2 Treg, highly suppressive Treg, activated Treg, or effector Treg cells in the sample is performed by measuring mRNA, protein, or both.

111. The method of claim 91, wherein the cancer vaccine is a tumor cell vaccine, an antigen vaccine, a dendritic cell vaccine, a DNA vaccine, an mRNA vaccine or a vector-based vaccine.

112. The method of claim 91, wherein the cancer vaccine is directed to a cancer antigen selected from at least one of: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, NY-ESO-1, LAGE-1, SSX-1, SSX-2 (HOM-MEL-40), SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RARalpha fusion protein, PTPRK, K-ras, N-ras, Triosephosphate isomerase, GnTV, Herv-K-mel, Lage-1, NA-88, /Lage-2, SP17, and TRP2-Int2, (MART-I), gp100, TRP-1, TRP-2, MAGE-1, MAGE-3, p15(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, mu-23H1, PSA, TAG-72-4, CA 19-9, CA 72-4, CAM 17.1, NuMa, K-ras, CDK4, Mum-1, p16, TAGE, PSMA, PSCA, CT7, telomerase, 43-9F, 5T4, 791Tgp72, alpha-fetoprotein, 13HCG, BCA225, BTAA, CA 125, CA 15-3, CA 195, CA 242, CA-50, CAM43, CD68\KP1, CO-029, FGF-5, G250, Ga733, HTgp-175, M344, MA-50, MG7-Ag, MOV18, NB\170K, NY-CO-1, RCAS1, SDCCAG16, TA-90, TAAL6, TAG72, TLP, TPS; or immunogenic fragments that comprise an epitope of any of the foregoing antigens.
